(19) United States
(12) Patent Application Publication
(10) Pub. No.: US 2016/0287211 A1
(43) Pub. Date: Oct. 6, 2016

(54) System and Method for Multi-Modal in Vivo Imaging

(71) Applicants: Ralph S. DaCosta, Etobicoke (CA); Brian C. Wilson, Toronto (CA); Israel Veilleux, Toronto (CA); Jin Ho Chang, Seoul (KR); Jeeun Kang, Seoul (KR); Yangmo Yoo, Gyeonggi-do (KR); Tai-Kyong Song, Seoul (KR)

(72) Inventors: Ralph S. DaCosta, Etobicoke (CA); Brian C. Wilson, Toronto (CA); Israel Veilleux, Toronto (CA); Jin Ho Chang, Seoul (KR); Jeeun Kang, Seoul (KR); Yangmo Yoo, Gyeonggi-do (KR); Tai-Kyong Song, Seoul (KR)

(21) Appl. No.: 15/085,380

(22) Filed: Mar. 30, 2016

Related U.S. Application Data
(60) Provisional application No. 62/140,919, filed on Mar. 31, 2015.

(51) Int. Cl. A61B 8/00; A61B 8/08; A61B 8/14

(52) U.S. Cl. CPC A61B 8/4416; A61B 8/14; A61B 8/4444; A61B 8/5261

(57) ABSTRACT

Various embodiments are described herein for a method, apparatus and system for performing multi-modal imaging using a common imaging probe having various modes of operation including a fluorescent (FL) imaging mode and at least one of an ultrasound (US) imaging mode, a photoacoustic (PA) imaging mode and a combined US/PA imaging mode. Molecular/functional information may be obtained from FL and PA imaging and anatomical information may be obtained from ultrasound (US) imaging. The system may be implemented to provide for images from each modality in real time as well as provide for co-registration of these images. Experimental results demonstrate that combining the imaging modalities does not significantly compromise the performance of each of the separate US, PA, and FL imaging techniques, while enabling multi-modality registration.

### CROSS-REFERENCE TO RELATED APPLICATION

This application claims the benefit of U.S. Provisional Patent Application No. 62/140,919 filed Mar. 31, 2015; the entire contents of Patent Application No. 62/140,919 are hereby incorporated by reference.

### FIELD

Various embodiments are described herein that generally relate to a system and method for tri-model in vivo imaging which use an integrated tri-modal imaging probe that can provide at least one of ultrasound (US), photoacoustic (PA) and fluorescent (FL) imaging.

### BACKGROUND

The use of in vivo multimodality imaging to acquire multifunctional information is becoming more widespread for preclinical research, clinical diagnosis, interventional guidance and treatment response monitoring.¹ As discussed by Marti-Bonmati et al.², separate modes can be acquired at different times on different imaging devices and then fused digitally or can be acquired synchronously using a single system with multi-modal capabilities. The latter facilitates image co-registration and minimizes changes in subject positioning, and in some cases allows simultaneous acquisition of data, which is valuable in imaging dynamic processes or in guiding real-time interventions such as surgery. For preclinical small-animal (typically mouse model) imaging there are several imaging systems available, including systems with up to 4 different modes (radioisotope, radiographic, fluorescence, luminescence). Other prototypes such as combined fluorescence tomography and single photon emission computed tomography/computed tomography (SPECT/CT) have also been reported.³

There is a similar trend in clinical imaging systems, with either fully integrated hybrid systems or with the modalities linked by common patient support/transfer.^{4,5} The objective is to efficiently collect images containing different biological information and retain accurate co-registration between them. In some applications anatomical information provided by CT or MRI scanning is used to correct for attenuation effects in PET or gamma-ray imaging, improving the accuracy of quantitative radionuclide uptake/biodistribution measurements. In the preclinical domain, there is a similar motivation to combine bioluminescence or fluorescence tomography with X-ray CT imaging in order to correct the optical modes for the light attenuation by intervening organs.⁶

In addition to hybrid optical and non-optical imaging,⁷ hybrid optical-optical imaging approaches are also increasingly reported and can significantly improve the sensitivity and/or specificity of tumor detection, especially for early cancer⁸: e.g. fluorescence plus optical coherence tomography,⁹ photoacoustic imaging plus optical coherence tomography,¹⁰ 3D bioluminescence plus diffuse optical tomographyll or fluorescence tomography plus diffuse optical tomography.¹²

### SUMMARY OF THE INVENTION

In a broad aspect, at least one embodiment described herein provides an imaging probe for obtaining image data of a region of interest (ROI) for a portion of an object using Fluorescence (FL) imaging and at least one acoustics-based imaging modality, wherein the imaging probe comprises a Fluorescent (FL) probe portion for obtaining FL image data of the ROI along a first plane at a certain depth of the object, the FL probe portion having a first longitudinal axis and being configured to deliver FL excitation energy from a first end of the imaging probe; and an acoustics probe portion for obtaining acoustics-based image data of the ROI along a second plane at various depths of the object, the acoustics probe portion being adjacent to the FL probe portion and having a second longitudinal axis that is parallel, co-planar and offset with respect to the first longitudinal axis of the FL probe portion and being configured to deliver excitation energy from the first end to evoke acoustic echoes from the ROI, wherein the FL image data and the acoustics-based image data can be obtained separately or synchronously with one another.

In at least one embodiment, the acoustics probe portion comprises an Ultrasound (US) transducer for delivering acoustic energy as the excitation energy and receiving the acoustic echoes for obtaining US image data as the acoustics-based image data.

In at least one embodiment, the acoustics probe portion comprises a light guide to deliver light energy as the excitation energy and an US transducer for receiving acoustic echoes generated by a Photoacoustic (PA) response from the ROI and the acoustics-based image data comprises PA image data.

In another broad aspect, at least one embodiment described herein provides a

In at least one embodiment, the acoustics probe portion is configured to deliver light energy, acoustic energy or light energy and acoustic energy as the excitation energy and to obtain PA image data, US image data or PA/US image data, respectively.

In at least one embodiment, the US transducer emits acoustic excitation energy at an acoustic focal depth that corresponds to the depth of the ROI and the light guide is bifurcated and comprises two output apertures disposed on either side of the US transducer that are angled to output two light beams that overlap at a PA focal depth that is similar to the acoustic focal depth taking into account dispersion of the light beams in the object.

In at least one embodiment, the FL probe portion comprises a light guide for delivering FL excitation light energy to the ROI, the light guide having a longitudinal axis that is parallel with a longitudinal axis of elements in the acoustics probe portion that deliver excitation energy to the ROI; a first optical path that comprises a light detector and zoom optics for obtaining the FL image data from the ROI at a desired magnification, the zoom optics being coupled to the light detector and having moveable lens elements; and a motor for actuating the moveable lens elements in the zoom optics for achieving the desired magnification.

In at least one embodiment, the FL probe portion further comprises a second optical path that comprises a second light detector and second zoom optics for obtaining additional FL image data from the ROI at the desired magnification to provide stereoscopic FL image data, the second zoom optics being coupled to the second light detector and having moveable lens elements that are controlled by the motor.

In at least one embodiment, a FL light source and optionally a White Light (WL) source is coupled to the light guide when obtaining the FL image data and optionally WL image data.

In at least one embodiment, the acoustics probe portion has an end portion that is adapted to contact a surface of the object during acoustics-based imaging and the FL probe portion has an end-portion with a stand-off relative to the end portion of the acoustic probe portion so as not to contact the surface of the object during FL imaging.

In at least one embodiment, the probe is portable and handheld.

In another broad aspect, at least one embodiment described herein provides a system for obtaining image data of a region of interest (ROI) for a portion of an object using Fluorescence (FL) imaging and at least one acoustics-based imaging modality, the system comprising a multi-modal imaging probe comprising an FL probe portion for obtaining FL image data of the ROI and an acoustics probe for obtaining acoustics-based image data of the ROI, the FL image data being obtained along a first plane at a first depth of ROI of the object and the acoustics-based image data being obtained along a plurality of second planes along various depths of the object about the ROI, the first and second planes having an angular relationship; and a processing unit for controlling the system to operate in various imaging modes of operation including a combined FL and acoustics-based imaging mode wherein the FL image data and the acoustics-based image data and portions of the acoustics-based image data obtained along the different second planes at the first depth are combined into multimodal image data by performing intermodal image registration.

In at least one embodiment, the system further comprises a mechanical scan system to move the probe relative to the object to obtain acoustics-based image data along the different second planes of the ROI or from different angles around the ROI.

In at least one embodiment, the processing unit is configured to generate at least one of reconstructed 2 dimensional (2D) and 3 dimensional (3D) PA and FL image data, combined US/PA image data and combined FL/PA/US image data.

In at least one embodiment, the processing unit is configured to provide one representation for a single sequence of multi-modal acquisition and spatio-temporal registration for a given FL, and US, PA or US and PA imaging sequence.

In at least one embodiment, the processing unit is configured to generate the multimodal image data by performing the intermodal image registration of the FL image data and one of US coronal plane (C-plane) image data, PA C-plane image data or combined US/PA C-plane image data.

In at least one embodiment, the combined US/PA C-plane image data are in multiple cross-sectional planes formed by lateral and axial axes, the FL image data are in a second C-plane at a given depth and the intermodal registration is based on using the multiple cross-sectional US/PA C-plane image data at the given depth to construct a C-plane US/PA image and onto which the FL image data is overlaid.

In at least one embodiment, the processing unit is configured to generate the combined US/PA C-plane image data by using alpha-blending.

In at least one embodiment, the processing unit is configured to apply pixel scaling when combining FL image data with US image data, PA image data or combined US/PA image data.

In at least one embodiment, the system may comprise FL and US/PA subsystems that are physically separate so that each imaging modality may be independently operated.

In at least one embodiment, the processing unit may be further configured to operate in an US imaging mode, a PA imaging mode, a FL imaging mode, or a combined US+PA imaging mode.

In another broad aspect, at least one embodiment described herein provides a method for obtaining image data of a region of interest (ROI) for a portion of an object, the method comprising positioning a multi-modal imaging probe with respect to the ROI; providing excitation light energy along a first longitudinal axis to the ROI to obtain Fluorescent (FL) image data from the ROI at the target of the object using the FL sensor, the FL image data obtained along a plane at a first depth of the ROI; providing excitation energy along a second longitudinal axis to the ROI to obtain acoustics-based image data from the ROI along a second plane for a plurality of depths of the ROI, the second longitudinal axis being collinear, coplanar and offset with the first longitudinal axis and the acoustics-based image data comprising Ultrasound (US) image data, Photoacoustic (PA) image data or US image data and PA image data; and combining the FL image data and the acoustics-based image data to generate multi-modal image data by performing intermodal image registration based on an orientation of the first and second planes and the offset.

In at least one embodiment, the method comprises obtaining the US image data or PA image data by translating the multi-modal imaging probe along an elevation (y) direction, for which 2D or 3D US imaging, 2D or 3D PA imaging or 2D or 3D US imaging is performed sequentially by moving the multi-modal imaging probe in increments.

In at least one embodiment, the method comprises generating the multimodal image data by performing the intermodal image registration of the FL image data and one of US coronal plane (C-plane) image data, PA C-plane image data or combined US/PA C-plane image data.

In at least one embodiment, the method comprises generating the combined US/PA C-plane image data by using alpha-blending.

In at least one embodiment, the method comprises applying pixel scaling when combining FL image data with US image data, PA image data or combined US/PA image data.

In at least one embodiment, the method comprises obtaining the multi-modal image data after a multi-modal contrast agent is applied to the object.

In another broad aspect, at least one embodiment provides a computer readable medium comprising a plurality of instructions that are executable on a microprocessor of an apparatus for adapting the device to implement a method for obtaining multi-modal image data of an object, wherein the method may be defined according to the teachings herein.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment and the drawings will now be briefly discussed.

FIG. 1A is a block diagram of an example embodiment of the overall configuration of a tri-modal imaging system that includes a tri-modal imaging probe, a combined Ultrasound (US)/PhotoAcoustic (PA) subsystem, a Fluorescence (FL) subsystem, and a mechanical scan system.

FIG. 1B is an example embodiment of the host process system shown in FIG. 1A.

FIG. 2A is a view of an example embodiment of an integrated tri-modal imaging probe that may be used with the tri-modal imaging system.

FIG. 2B is a 3D CAD drawing of an example embodiment of an integrated tri-modal imaging probe that may be used with the tri-modal imaging system.

FIG. 2C is an expanded view of an example embodiment of the FL subsystem of the integrated tri-modal imaging probe.

FIG. 2D is an example of the optical design of the zoom function that may be used with the FL subsystem in 2 positions (1× and 5×).

FIG. 2E is an example of a US/PA subsystem that may be used with the integrated tri-modal imaging probe along with a zoomed-in view of the US/laser delivery section (on the right side of the figure).

FIG. 3 is block diagram of an example embodiment of a combined US/PA subsystem that may be used in the integrated tri-modal imaging probe.

FIG. 4 is a block diagram of an example embodiment of an FL subsystem showing LED light sources, cameras, a zoom stepping motor/drive and the control electronics, which can be used with the integrated tri-modal imaging probe.

FIG. 5 is a flow chart of an example embodiment of a tri-modal imaging method with a tri-modal imaging probe.

FIG. 6A-6C are normalized laser beam profiles for PA imaging along the axial, lateral and elevated axes, respectively showing axial, lateral and elevated distributions at the acoustic focal plane (7 mm). The dotted line in the lateral beam profile indicates the image width of the US/PA subsection.

FIGS. 7A and 7B show the pulse-echo response and the frequency spectrum, respectively, of the US linear array transducer.

FIG. 8A shows an example PA image of a 0.3 mm pencil core made of graphite.

FIGS. 8B and 8C show examples of the corresponding intensity profile of the pencil core along the lateral and axial directions, respectively.

FIG. 9A is an example of images taken with the FL subsystem under white light (left image) and fluorescence mode (right image) for vials containing 1 and 2 μM concentrations of PpIX (single frame, 100 ms integration).

FIG. 9B is an example of images taken with the FL subsystem using stereo imaging in reflectance mode where the images include a left view, an anaglyph and a right view.

FIG. 9C is an example of in vivo images taken with the FL subsystem under ambient light conditions of a mouse bearing a subcutaneous tumor, following intratumoral PpIX injection (single frame, 100 ms integration), showing the reflectance and fluorescence images and their combination.

FIGS. 10A-10D shows examples of reconstructed multi-modal images of overlapping fluorophore-filled tubes at depth in a water phantom: (10A) white-light, (10B, 10C) FL and the corresponding US and (10D) PA C-plane images overlaid on the FL image in which mb stands for methylene blue and fl stands for fluorescein, respectively.

FIGS. 11A-11B show examples of reconstructed in vivo images before and after, respectively, intratumoral injection of a contrast mixture of 50:50 of methylene blue and fluorescein.

Further aspects and features of the embodiments described herein will appear from the following description taken together with the accompanying drawings.

### DETAILED DESCRIPTION OF THE INVENTION

Various apparatuses or processes will be described below to provide an example of an embodiment of the claimed subject matter. No embodiment described below limits any claimed subject matter and any claimed subject matter may cover processes or apparatuses that differ from those described below. The claimed subject matter is not limited to apparatuses, devices, systems or processes having all of the features of any one apparatus, devices, systems or process described below or to features common to multiple or all of the apparatuses, devices, systems or processes described below. Any subject matter disclosed in an apparatus, device, system or process described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures and components have not been described in detail so as not to obscure the example embodiments described herein. Also, the description is not to be considered as limiting the scope of the example embodiments described herein in any way, but rather as merely describing the implementation of various embodiments as described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate a time interval between electrical stimulation impulses. “Tight coupling” or “Closely coupled” as used herein mean a relatively short time interval between such impulses.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

In addition, as used herein, the wording “and/or” is intended to represent an inclusive-or. That is, “X and/or Y” is intended to mean X or Y or both, for example. As a further example, “X, Y and/or Z” is intended to mean X or Y or Z or any combination thereof.

Described herein are various example embodiments of a device that may be used for multi-modality imaging, which is beneficial for both preclinical and clinical applications as it enables complementary information from each modality to be obtained in a single procedure. For example, the design, fabrication and testing of a novel multi-modal in vivo imaging system to exploit molecular/functional information from fluorescence (FL) and photoacoustic (PA) imaging as well as anatomical information from ultrasound (US) imaging is described herein. The embodiments described herein involve the use of FL imaging and at least one acoustics-based imaging modality being US imaging, PA imaging or a combination of US and PA imaging.

For example, in one example embodiment, a compact probe comprises an ultrasound transducer that may be used for both US and PA imaging. In addition, a pulsed laser light may be brought into the compact probe by fiber-optic bundles. The FL subsystem may be independent of the acoustic components but the front end that delivers and collects the light may be physically integrated into the same probe.

In addition, an example embodiment is described herein for a multi-modal imaging system that may be implemented to provide each modality image in real time as well as co-registration of the images. The performance of such a system was evaluated through phantom and in vivo animal experiments. The results demonstrate that combining the modalities does not significantly compromise the performance of each of the separate US, PA, and FL imaging techniques, while enabling multi-modality registration. The potential applications of this novel approach to multi-modality imaging range from preclinical research to clinical diagnosis, especially in detection/localization and surgical guidance of accessible solid tumors.

The tri-modal imaging system may be used to exploit the complementary information from each modality in a single procedure, as summarized in Table 1 with reference specifically to in vivo oncology applications. Thus, US imaging, which is the most commonly used imaging modality in clinics, allows for non-invasive real-time structural/functional imaging deep within tissue while functional blood flow information can be obtained using Doppler techniques.

PA imaging has emerged in the last few years and is itself a hybrid between optical and acoustic technologies.¹³ Incident light in the form of short (˜10 ns) laser pulses, usually in the visible/near-infrared range, is elastically-scattered by the tissue and is thereby spatially distributed throughout the volume of interest. Local absorption of the light energy leads to a transient small increase in temperature that induces local thermoelastic expansion, generating acoustic waves that are detected at the tissue surface to construct an ultrasound image of the distribution of optical absorbers. Thus, PA imaging uses the molecular specificity of light absorption and the imaging depth capability of ultrasound, which also defines the spatial resolution of the images.

TABLE 1
Comparison of ultrasound, photoacoustic and fluorescence imaging
with reference to in vivo cancer imaging

| | Ultrasound | Photoacoustic | Fluorescence |
|---|---|---|---|
| Image contrast | Acoustic impedance | Optical absorption and | Optical absorption and |
| mechanism | mismatch and | conversion to acoustic | re-emission |
| | acoustic scattering | wave | |
| Information | Organ/tissue | (Micro)vasculature | Endogenous |
| content | structure | | fluorophore content |
| | | | from autofluorescence |
| | Blood flow | Intravascular Hb, SO₂ | Uptake of exogenous |
| | | | fluorophores |
| Typical | 30 cm and 300 μm | 2-5 cm and 800 μm | <1-2 mm, <<100 μm for |
| Imaging depth | at 5 MHz | for 5 MHz acoustic | wide field imaging |
| and spatial | 1 cm and 100 μm at | wave detection | |
| resolution | 50 MHz | | |
| Image format | B-mode (2d section perpendicular to the | | En-face to the tissue |
| | tissue surface) or C-mode (parallel to the | | surface |
| | tissue surface) | | |
| Exogenous | Microbubbles | Molecular dyes or | Molecular dyes, |
| contrast | | nanoparticles with | activatable beacons, |
| agents/ | | high optical absorption | fluorescent |
| Tumor- | Not standard | Yes | nanoparticles |
| targetable? | | | Yes |
| Main uses in | Tumor detection | Not yet established | Detection of early |
| clinical | Interventional | | cancers/dysplasias |
| oncology | guidance | | Surgical guidance |
| currently | | | |

In the absence of an administered (optical) contrast agent, the primary absorber in tissue in the visible and near-infrared wavelength range around 500-850 nm is hemoglobin, and wavelength scanning enables separation of the Hb and HbO₂ contributions based on their different absorption spectra, thereby allowing images to be determined of the important functional parameter of oxygen saturation, SO₂=[HbO₂]/{[Hb]+[HbO₂]}. Exogenous contrast agents for PA imaging, either targeted or untargeted, have been reported, based on dyes or nanoparticles with high light absorption.¹⁴

The image format and spatial resolution is essentially the same as for ultrasound imaging. However, the contrast is optical in nature and the maximum depth of imaging is determined mainly by the attenuation of the laser light. However, unlike other purely optical techniques, the high scattering of light by tissue is an advantage, since it distributes the energy and allows volumetric images to be created to depths of several cm. The lateral extent of the images is determined primarily by the width of the optical field. Although PA imaging has been used widely in small animal imaging, it is only now starting to penetrate into clinical applications.¹⁵

The third modality, FL imaging, is purely optical. In most clinical applications visible or near-infrared excitation light is used and the fluorescence is detected from the same tissue surface as the incident excitation light. Due to high light scattering by tissue, the excitation and emission light are strongly attenuated and there is no depth discrimination unless special techniques (e.g. confocal, tomographic) are used. The endogenous (auto)fluorescence of the tissue can be imaged and this has shown high sensitivity for early cancer/pre-cancer detection in different sites.¹⁷ Alternatively, there are many exogenous fluorophores: molecular dyes, activatable molecular beacons, fluorescence nanoparticles and, for preclinical use, fluorescent proteins.¹⁸ The advantage of fluorescence imaging is the high biological specificity through the use of targeted fluorophores. Real-time fluorescence imaging is used for surgical guidance, particularly to identify tumor margins that are not visible in pre-operative radiological imaging.¹⁹

Hence, these 3 modalities have complementary strengths and limitations. Hybrid US plus PA imaging may use a common acoustic transducer and electronics for performing transmitting and receiving of US, PA or both US and PA. No devices are known that use a tri-modal combination of US, PA and FL imaging. In one example embodiment, the PA and FL modes may be built onto an established medical ultrasound platform through design and fabrication of a novel tri-modal handpiece.

A tri-modal imaging system that uses a combination of US, PA and FL imaging may have a variety of uses such as, but not limited to, surgical guidance, for example. In the case of surgical guidance, the volumetric US images may provide the overall anatomical context/localization, and the PA images may provide better volumetric tumor contrast through the altered microvasculature. These images may be used prior to and during bulk resection of solid tumor while the FL imaging may be used near the end of surgery in order to detect any residual tumor that is not seen either on pre-operative radiological images or intra-operatively with PA imaging. Lymph nodes may also be evaluated for the presence of tumor cells.

In some example embodiments, tri-modal contrast agents may be used with the tri-modal imaging system. For example, several multifunctional contrast agents have been reported, e.g. for combined PET and florescence,²¹ that may be used with tri-modal imaging for at least one of the embodiments described in accordance with the teachings herein. The tri-modal contrast agents may include microbubbles formed from porphyrin-lipid complexes. The intact microbubbles have strong optical absorption due to their high porphyrin content that provides the PA contrast, while the intrinsic FL of the porphyrin component provides the FL contrast. Tri-modal in vivo imaging in a murine tumor model has recently been shown with these microbubbles using a bench-top US/PA imager and separate whole-body small-animal fluorescence imaging system.²³

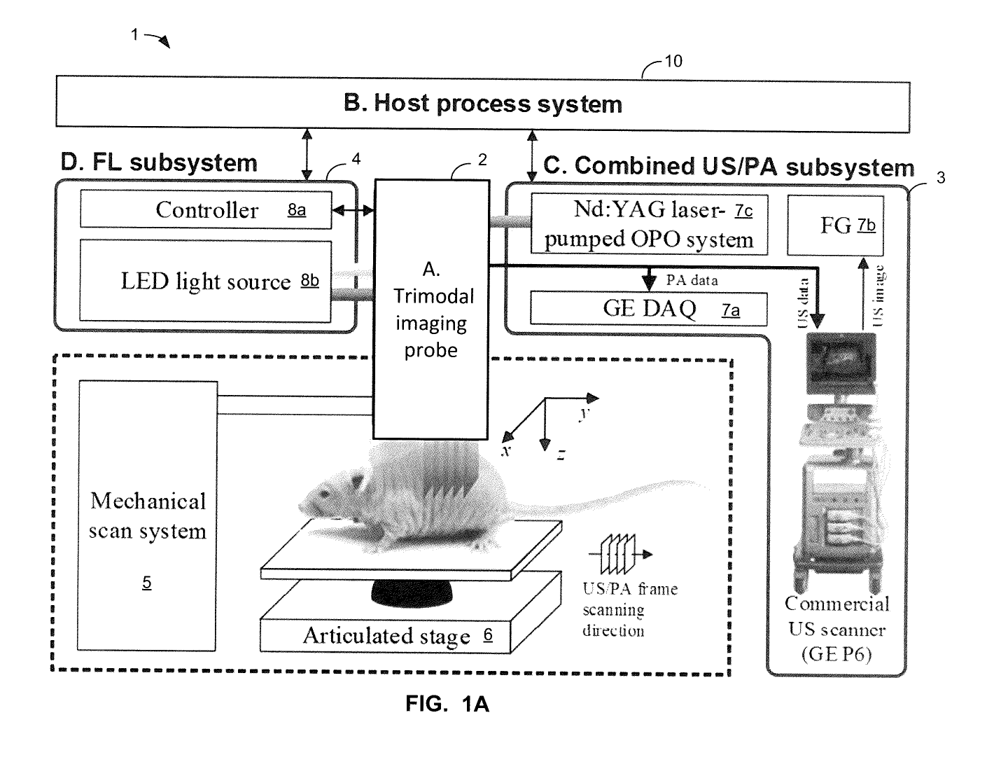

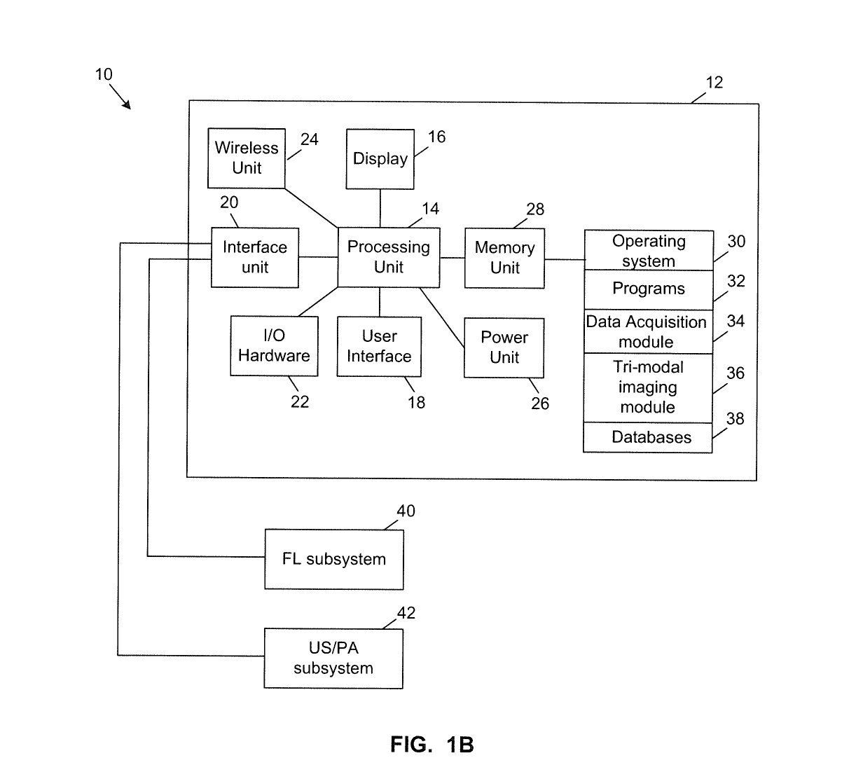

Referring now to FIG. 1A, shown therein is an overall block diagram of an example embodiment of a prototype tri-modal imaging system 1. The tri-modal imaging system 1 generally includes a tri-modal probe 2, an US and PA dual-modality subsystem 3, an FL subsystem 4 and a mechanical scan system 5. The tri-modal probe 2 may be mounted on the mechanical scan system 5 for small animal imaging as shown in FIG. 1A or may be handheld for clinical use. The object, a portion of which is being imaged, may be placed on an articulated stage 6. There may be alternative embodiments in which the US and PA dual-modality subsystem 3 may be replaced by an US modality subsystem or a PA modality subsystem.

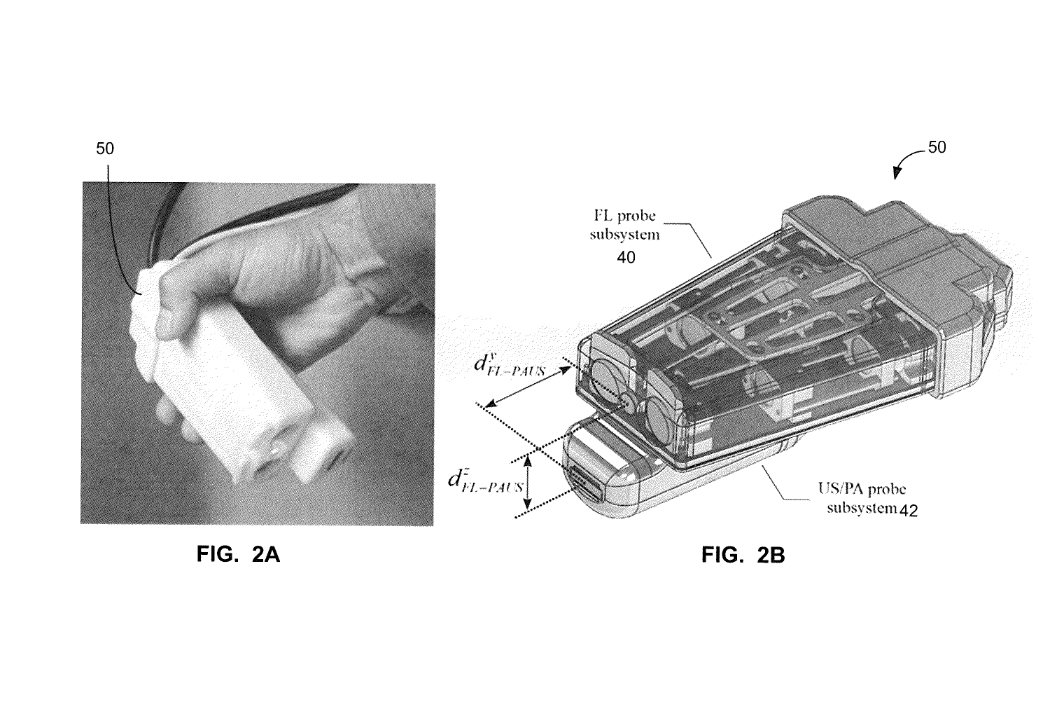

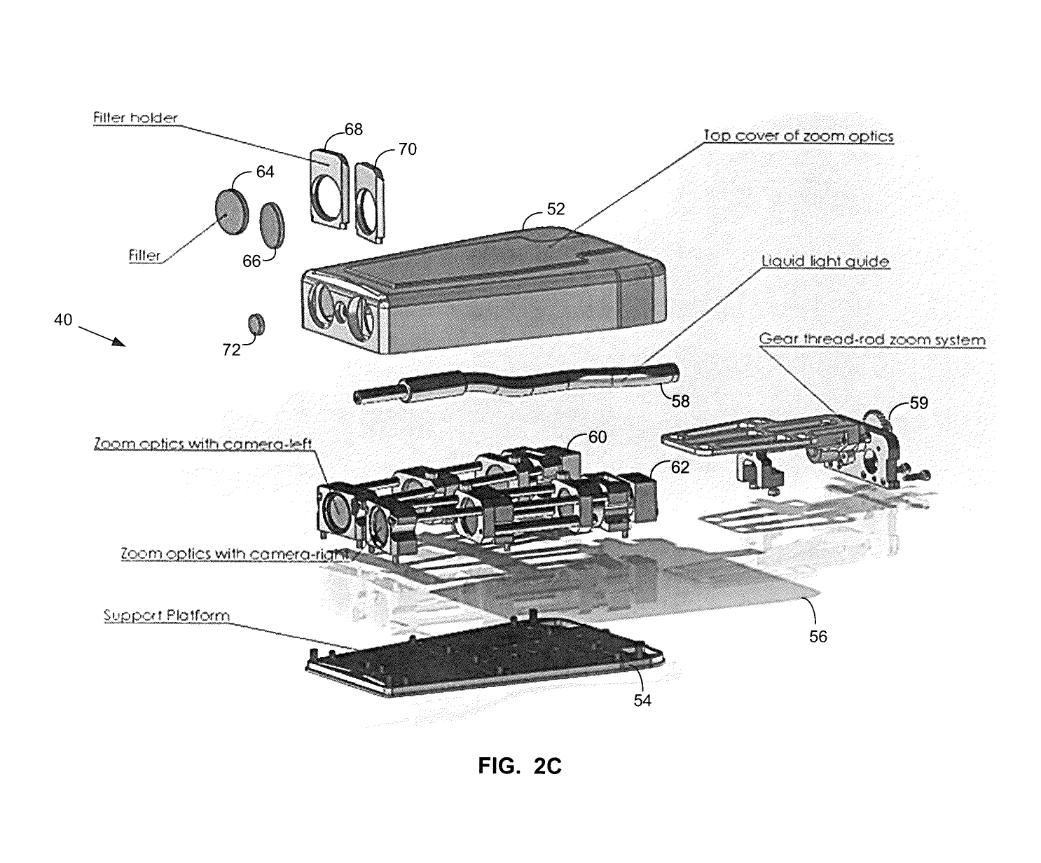

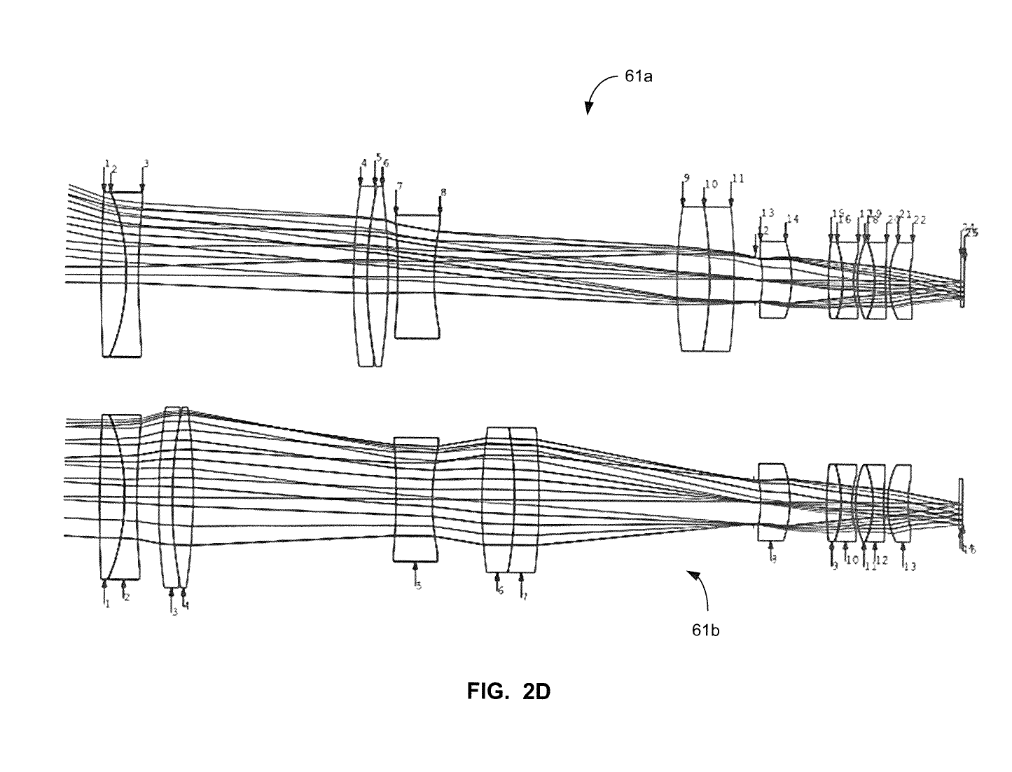

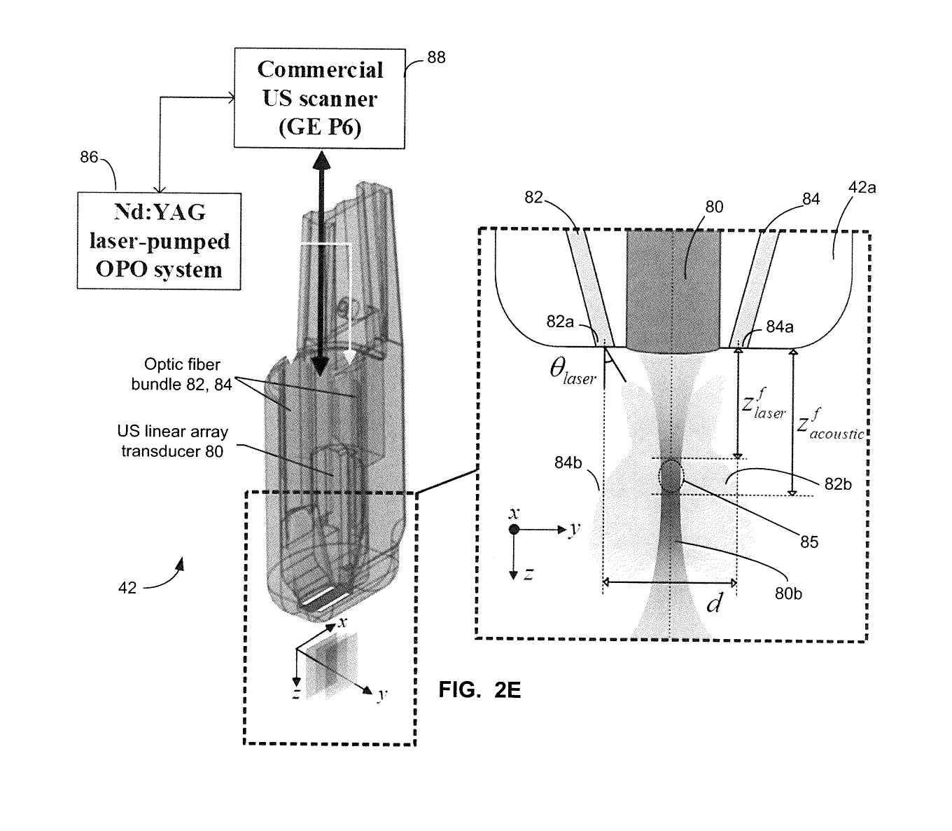

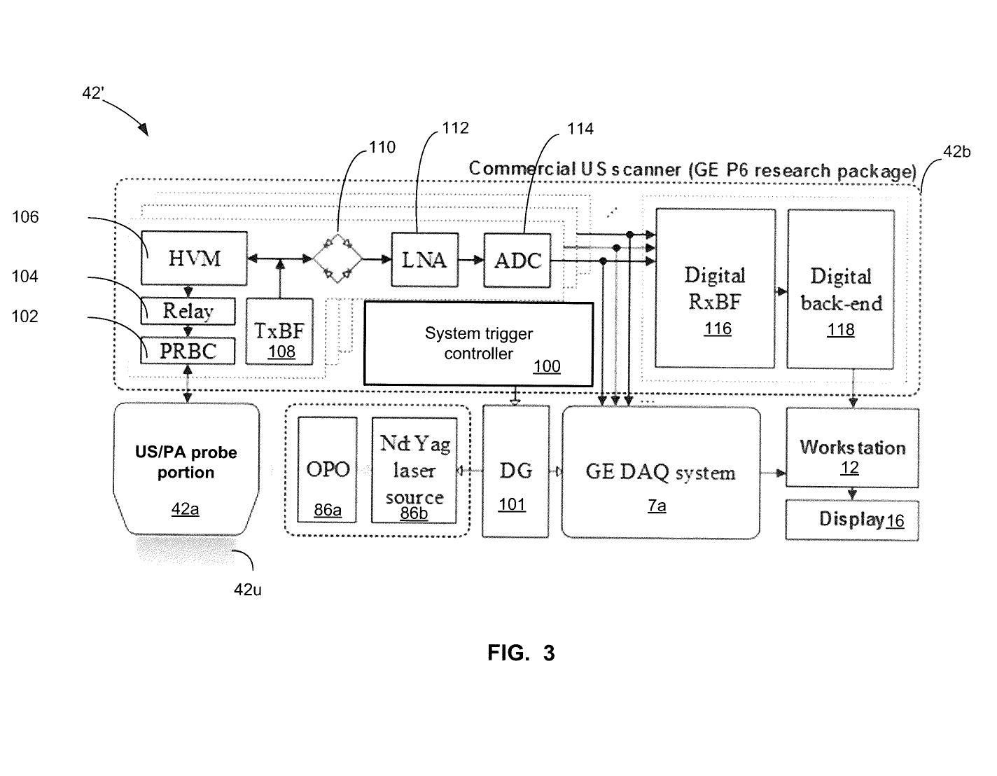

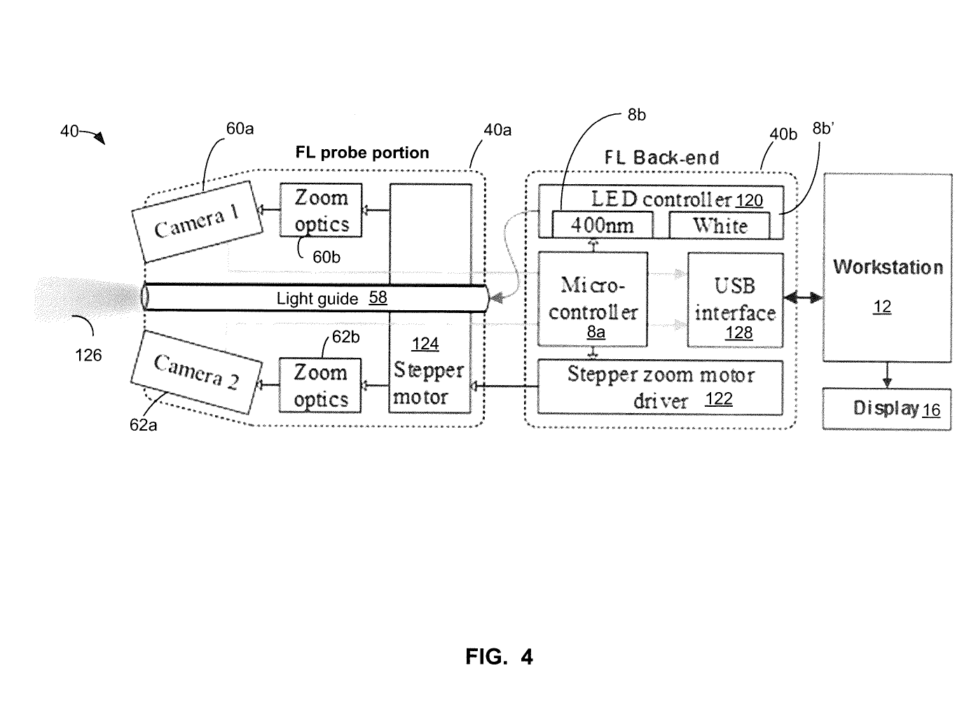

A host process may be used to control all of the subsystems, either operating in separate US, PA, or FL modes, combined US+PA modes or a combined FL+PA+US mode. The combined US and PA subsystem 3 may comprise a GE Data Acquisition (DAQ) module 7a, a Frame Grabber (FG) 7b, a PA excitation source 7c and an US excitation source (not shown). The GE DAQ and FG modules 7 and 8 may be implemented using the GE p6 US research package. The PA excitation source 7c may be implemented using a laser, such as a YAG laser-pumped OPO system (described in more detail with regards to FIGS. 2A, 2E and 3). The PA and US excitation sources deliver energy to a portion of the object to be imaged to evoke acoustic energy from which acoustic-based image data is obtained. The FL subsystem 4 may comprise a controller 8a, an LED light source, a light guide (not shown) and at least one light detector (not shown) to provide light excitation to the portion of the object to be imaged and to detect light emitted by the portion of the object in response to the light excitation signal. Example embodiments of the FL subsystem are described in more detail in FIGS. 2A-2E and 4.

The images in the stand-alone modes may be updated at maximum frame rates of 15 (FL), 10 (PA) and 145 (US) frames per second (fps). In US and PA mode, the frame rates may be unchanged when they are combined (US+PA). Otherwise, the tri-modal imaging mode may be configured to provide one image for a single sequence of tri-modal acquisition and spatio-temporal registration for a given US, PA or FL imaging sequence. It should be noted that in other embodiments, other types of data acquisition hardware and software may be used where possible.

Referring now to FIG. 1B, shown therein is a block diagram of an example embodiment of the host process system 10 that can be used to perform tri-modal imaging. The system 10 includes an operator unit 12, an FL subsystem 40, and an US/PA subsystem 42. The system 10 further includes several power supplies (not all shown) connected to various components of the system 10 as is commonly known to those skilled in the art. In general, a user may interact with the operator unit 12 to obtain at least one image using one or more imaging modalities. The system 10 is provided as an example and there can be other embodiments of the system 10 with different components or a different configuration of the components described herein. In alternative embodiments, the US/PA subsystem 42 may be replaced by an US subsystem or a PA subsystem.

The operator unit 12 comprises a processing unit 14, a display 16, a user interface 18, an interface unit 20, Input/Output (I/O) hardware 22, a wireless unit 24, a power unit 26 and a memory unit 28. The memory unit 28 comprises software code for implementing an operating system 30, various programs 32, a data acquisition module 34, a tri-modal imaging module 36 and one or more databases 38. Many components of the operator unit 12 can be implemented using a desktop computer, a laptop, a mobile device, a digital tablet, and the like.

The processing unit 14 controls the operation of the operator unit 12 and can be any suitable processor, controller or digital signal processor that can provide sufficient processing power processor depending on the configuration, purposes and requirements of the system 10 as is known by those skilled in the art. For example, the processing unit 14 may be a high performance general processor. In alternative embodiments, the processing unit 14 can include more than one processor with each processor being configured to perform different dedicated tasks. In alternative embodiments, specialized hardware can be used to provide some of the functions provided by the processing unit 14.

The display 16 can be any suitable display that provides visual information depending on the configuration of the operator unit 12. For instance, the display 16 can be a cathode ray tube, a flat-screen monitor and the like if the operator unit 12 is a desktop computer. In other cases, the display 16 can be a display suitable for a laptop, tablet or handheld device such as an LCD-based display and the like depending on the particular implementation of the operator unit 12.

The user interface 18 can include at least one of a mouse, a keyboard, a touch screen, a thumbwheel, a track-pad, a track-ball, a card-reader, voice recognition software and the like, again depending on the particular implementation of the operator unit 12. In some cases, some of these components can be integrated with one another.

The interface unit 20 can be any interface that allows the operator unit 12 to communicate with other devices, computers or systems. In some embodiments, the interface unit 20 can include at least one of a serial bus or a parallel bus. The busses may be external or internal. The busses may be at least one of a SCSI, USB, IEEE 1394 interface (FireWire), Parallel ATA, Serial ATA, PCIe, or InfiniBand. The host interface component 134 may use these busses to connect to the Internet, a Local Area Network (LAN), a Wide Area Network (WAN), a Metropolitan Area Network (WAN), a Wireless Local Area Network (WLAN), a Virtual Private Network (VPN), or a peer-to-peer network, either directly or through a modem, router, switch, hub or other routing or translation device. Various combinations of these elements can be incorporated within the interface unit 20.

The I/O hardware 22 is optional and can include, but is not limited to, at least one of a microphone, a speaker and a printer, for example.

The wireless unit 24 is optional and can be a radio that communicates utilizing CDMA, GSM, GPRS or Bluetooth protocol according to standards such as IEEE 802.11a, 802.11b, 802.11g, or 802.11n. The wireless unit 24 can be used by the operator unit 12 to communicate with other devices or computers.

The power unit 26 can be any suitable power source that provides power to the operator unit 12 such as a power adaptor or a rechargeable battery pack depending on the implementation of the operator unit 12 as is known by those skilled in the art.

The memory unit 28 includes volatile and non-volatile computer storage elements such as, but not limited to, one or more of RAM, ROM, one or more hard drives, one or more flash drives or some other suitable data storage elements such as disk drives, etc. The memory unit 28 may be used to store an operating system 30 and programs 32 as is commonly known by those skilled in the art. For instance, the operating system 30 provides various basic operational processes that are used during the operation of the operator unit 12. The programs 32 include various user programs to allow a user to interact with the operator unit 12 to perform various functions such as, but not limited to, viewing and manipulating data and/or sending messages.

The data acquisition module 34 may be used to obtain image data from an object such as a patient or subject, which may be done under various imaging modes as described previously. Accordingly, the data acquisition module 34 may also be used to control the timing for data acquisition as well as setting various acquisition parameters such as the image acquisition rate, gain settings and white balance factors, while also receiving the pixel data stream.

The tri-modal imaging module 36 processes the image data that are acquired by the data acquisition module 34 in order to provide an image under a given imaging modality or to create multimodal images by combining image data obtained using different imaging modalities. For example, the tri-modal imaging module 36 may perform 3D reconstruction in at least some embodiments. This may include US and PA C-scan (i.e. volumetric) images as well as corresponding 3D stereoscopic FL imaging. The generated image data can then be provided as an output consisting of an electronic file or a display image with information in the form of an US, PA or FL image or combined image data as described herein or in another suitable form for conveying information in the obtained images.

In alternative embodiments, modules 34 and 36 may be combined or may be separated into further modules. The modules 34 and 36 are typically implemented using software, but there may be instances in which they are implemented using FPGA or application specific circuitry.

The databases 38 may be used to store data for the system 10 such as system settings, parameter values, and calibration data. The databases 38 may also be used to store other information required for the operation of the programs 32 or the operating system 30 such as dynamically linked libraries and the like.

The operator unit 12 comprises at least one interface that the processing unit 14 communicates with in order to receive or send information. This interface can be the user interface 18, the interface unit 20 or the wireless unit 24. For instance, information for obtaining images under one or more of the modes may be inputted by someone through the user interface 18 or it may be received through the interface unit 20 from another computing device. The processing unit 14 can communicate with either one of these interfaces as well as the display 16 or the I/O hardware 22 in order to output information related to the tri-modal imaging. In addition, users of the operator unit 12 may communicate information across a network connection to a remote system for storage and/or further analysis. This communication can include, but is not limited to, email, text or MMS communication, for example.

A user may also use the operator unit 12 to provide information needed for system parameters that are needed for proper operation of the tri-modal imaging system such as calibration information and other system operating parameters as is known by those skilled in the art. Data that is obtained from tests, as well as parameters used for operation of the system 10, may be stored in the memory unit 28. The stored data may include raw sampled data as well as processed cardiac map data.

At least some of the elements of the system 10 that are implemented via software may be written in a high-level procedural language such as object oriented programming and/or a scripting language. Accordingly, the program code may be written in C, C⁺⁺, MATLAB, JAVA, SQL or any other suitable programming language and may comprise modules or classes, as is known to those skilled in object oriented programming. Alternatively, or in addition thereto, some of the elements of the system 10 that are implemented via software may be written in assembly language, machine language or firmware as needed. In either case, the language may be a compiled or an interpreted language.

At least some of the program code can be stored on a computer readable medium such as, but not limited to, ROM, a magnetic disk, an optical disc or a computing device that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

Furthermore, at least some of the components described herein are capable of being distributed in a computer program product comprising a computer readable medium that bears computer usable instructions, such as program code, for one or more processors. The medium may be provided in various forms, including non-transitory forms such as, but not limited to, one or more diskettes, compact disks, tapes, chips, USB keys, external hard drives and magnetic and electronic storage. In alternative embodiments, the medium may be transitory in nature such as, but not limited to, wire-line transmissions, satellite transmissions, Internet transmissions or downloads, digital and analog signals, and the like. The computer useable instructions may also be in various forms, including compiled and non-compiled code.

### A. Tri-Modal Imaging Probe

US and PA imaging involve contacting the imaging probe with the tissue surface using an acoustic coupling medium such as a gel, a gel pad or water. This may not be desirable for FL imaging since, although the acoustic coupling media are optically transparent, the presence of significant thicknesses in the light field may distort the FL images. An example of a tri-modal imaging probe 50 is shown in FIGS. 2A-2E. This probe design allows the US/PA transducer to contact the tissue, while the FL subsection has a stand-off from the tissue surface defined by the parameter d_{FL-PAUS}^{y} which denotes the axial distance between the front faces of the FL and US/PA elements and the parameter d_{FL-PAUS}^{z} which is the elevation distance between the centers of the FL and US/PA elements. Note that the axial direction is parallel to the optical and acoustic beam propagation direction, the lateral direction is perpendicular to the axial direction, while the elevation direction is perpendicular to the US/PA imaging plane formed by the axial and lateral axes.

The imaging probe may be attached by a single cable housing (not shown) (e.g. 15 mm diameter) that holds the electrical cables for the US transducer, the optical fibers to deliver the PA laser pulses, the optical fiber bundles to deliver the FL excitation light, the electrical power and signal cables for the FL cameras and its mechanized optical zoom, as well as data wires for sending US, PA and/or FL image data, depending on the imaging mode, to post-processing stages that may be performed by a computing device that is coupled to the probe 50. In some embodiments, the FL subsection 40 and the US/PA subsections 42 of the imaging probe 50 may be physically separated, so that each imaging mode can be independently operated if required. The probe shell includes a top cover 52 and a support platform 54 that may both be fabricated by using stereolithographic 3D printing of photo-curable resin (Accura 25: 3D Systems Europe Ltd., Hemel Hempstead, UK). The overall shell dimensions of an example embodiment of the tri-modal imaging probe 50 are: 17.0 cm in length, 8.5 cm in width and 5.5 cm in thickness. The total weight, excluding the cable, is 293 g.

An example embodiment of the fluorescence subsection 40 is shown in FIGS. 2C, 2D, 2E and 4. This example embodiment has a custom-designed (Isbrucker Consulting Inc., ON, Canada) stereo-and-zoom optical configuration using off-the-shelf optical components. Each of the 2 optical paths 60 and 62 are along optical axes that converge. The optical paths 60 and 62 may each comprise a 120 mm long, 15 mm diameter compound lens with various lens elements with a 1× to 5× zoom driven by a miniature stepper motor 124 (Micromo, FL, USA). FIG. 2D shows the positions of various lenses for a first configuration 61a to achieve a first amount of zoom and a second configuration 61b to achieve a second amount of zoom which is achieved by the stepper motor 124 and a gear thread-rod zoom system 59 that engages certain moveable lens elements in the optical paths 60 and 62 for changing the position of these moveable lens elements in the optical paths 60 and 62 depending on the magnification used to obtain the FL image data. The zoom switch time was approximately 5 s, which was dictated by the stepping motor 124. The F/4.0 imaging optics gave fields of view of 8 cm and 1.6 cm diameter for the 1× and 5× zoom, respectively, at a nominal working distance of 12 cm. The angle between the optical axes was about 15° so that the images obtained from each optical path overlapped fully at the working distance. Two 5.7 mm×4.3 mm detectors 60a and 62a that may be used for the two stereoscopic angles are 2592×1944 pixel 12-bit MT9P031 CMOS arrays (Aptina, CA, USA) with integrated Bayer color filters 64 and 66, which may be held in place by filter holders 68 and 70 respectively. A center filter 72 may be used for the excitation light that is provided by the FL subsystem 40 to the portion of the object that is to be imaged. In some embodiments, color cameras may be used in order to have true-color capability in white-light mode, which provides the tissue/anatomical context for interpreting the corresponding FL images at the same location. Bayer filters 64 and 66 may be used which cause a slight loss in sensitivity (˜10% at around 650 nm at which the FL imaging was performed). The CW excitation light source may be provided by two high-power collimated LEDs (Mightex Systems, ON, Canada) including a white light source 8b′ for reflectance imaging (warm white 4000K, ˜100 mW delivered power) and a 400±10 nm light source 8b for FL excitation (˜450 mW). The CW excitation light source may be coupled to a 3 mm core, liquid light guide 58 (Thorlabs Inc., Newton, N.J., USA), the distal end of which may be centered on the imaging probe 50 between the two imaging axes and may be implemented to have a numerical aperture of 72°. The user can switch rapidly between broad-band white light for visualizing the tissue surface and blue light for fluorescence that is detected through a 450 nm long-pass filter with >90% in-band and 10⁻⁴ out-of-band transmission (Edmund Optics, Barrington, N.J., USA). The camera full-frame read-out rate was up to 5 Hz but with 4×4 binning this may increase to 15 Hz but may be limited by the speed of the USB read-out ports.

Referring now to FIG. 2E, for PA and US imaging, the spatial resolution was determined primarily by the center frequency of the transducer 80. Since this prototype system may be used for applications requiring an imaging depth of a few cm, a 15-MHz 128-element linear array transducer 80 was designed and fabricated. To improve the acoustic signal sensitivity, PMN-PT single crystals may be used as the active material²⁴ for the transducer 80. The geometrical specifications for an example embodiment of the transducer 80 are summarized in Table 2.

TABLE 2
Specs of the linear array transducer for combined US/PA imaging

| Subject | Unit | Specification |
|---|---|---|
| Number of elements | EA | 128 |
| Element pitch | mm | 0.1 |
| Element height | mm | 1.5 |
| Element kerf size | mm | 0.02 |
| Geometric lens focal depth | mm | 7 |

PA signals may be induced following a series of 7 ns laser pulses and the delivery optics (fiber optic bundles 82 and 84) may be integrated within the acoustic transducer section 42a, for example as shown in FIG. 2E, such that the excitation light beams 82b and 84b from the fiber optic bundles 82 and 84 may be distributed uniformly and overlap at a target tissue volume 85 which is in the portion of the object that is to be imaged. This may be achieved by using a custom-made bifurcated optical fiber bundle 82 and 84 with 50 μm fibers having a 0.55 numerical aperture (Fiberoptic Systems, Inc., Simi Valley, Calif., USA). The output apertures 82a and 84a of the fiber optic bundles 82 and 84 may be configured as 0.89 mm×13 mm rectangles. The input end of the fiber bundle (i.e. the end opposite the bifurcated end) had a 9.5 mm diameter, which was identical to the spot size of the Nd: YAG laser-pumped OPO system 86 (Surelite III-10 and Surelite OPO Plus, Continuum Inc., Santa Clara, Calif., USA), allowing the source laser beam to be coupled directly into the fiber bundle. The bifurcated outlets 82a and 84a may be placed on each side of the US transducer 80, with a separation distance of d=5.95 mm and may be tilted inwards at 30° (θₗₐₛₑᵣ), so that the depth of the center of the laser beams 82b and 84b overlap at a PA focal depth at a distance in air at zₗₐₛₑᵣ^{f} (e.g., see FIG. 2E) where zₗₐₛₑᵣ^{f} may be given by equation 1.

$${z}_{laser}^{f} = \frac{d}{2 tan {θ}_{laser}} . \qquad ( 1 )$$

Hence, zₗₐₛₑᵣ^{f} of the US/PA subsection was 5.15 mm in this example embodiment, which was slightly shorter than the geometrical acoustic focal depth, z_{acoustic}^{f}=7.00 mm in region 85 of the ultrasound energy from the ultrasound transducer 82. Since the laser beam diffuses as it propagates through the tissue, it was expected that the resulting overlap at 85 may match with the acoustic focal area 82, and this was experimentally verified.

### B. Host Process System

Referring again to FIG. 1, in an example embodiment, the host process system 10 may comprise a workstation 12, a Graphical User Interface (GUI) 18 and a monitor 16, as summarized in Table 3. The host process system 10 may be configured to control the tri-modal imaging system 1, to reconstruct the PA and FL images, and to combine either US/PA or FL/PA/US images. Depending on the imaging mode, the mechanical scan system 5 may also be controlled to translate the tri-modal imaging probe 2. The light sources for FL and PA imaging and their operating parameters may also be selected in some embodiments using the host process system 10.

TABLE 3
Specifications of the host process system for PA image
reconstruction and tri-modal registration

| Category | Description |
|---|---|
| Operating system (OS) | Windows 7 Professional 64 bit |
| Central processing unit (CPU) | Intel i7-3 3770K |
| Random access memory (RAM) | DDR3 16G PC3-19200 CL10 |
| Graphic processing unit (GPU) | Nvidia Geforce GTX690 |
| | PCI Express 3.0 (<16 GB/sec) |
| | CUDA cores: 3072 at 915 MHz |
| | 4096 MB GDDR3 |
| | (memory BW: 384 GB/s) |

A Compute-Unified Device Architecture (CUDA)-based parallel processing software may be used for PA image reconstruction. This may include dynamic beamforming²⁵ and back-end processing²⁶, which consumed about 9.9 ms of overall processing time for a 4 cm imaging depth based on the experimental setup. This corresponded to a frame rate of 101 Hz, which was sufficient to complete the reconstruction at the current frame rate (10-20 Hz); the latter is mainly determined by pulse repetition frequency of the laser (e.g. 10 Hz). On the other hand, the US images may be reconstructed using a commercial US scanner (such as the Logiq P6 system, GE Healthcare, Gyeonggi-do, Korea) and may be received by the host processing system 10 through the frame grabber 7b (e.g. a VGA2USB LR, Epiphan Systems Inc., Ottawa, Ontario, Canada). In FL mode, the raw pixel values from the CMOS cameras were read out through a USB port and saved as 8-bit RGB images. For white-light reflectance, the images were white-balanced by calibrating against a reference standard. The fluorescence and white-light images may be displayed in true color.

Each of the FL image data and separate US and PA image data, or alternatively combined US/PA image data, may be displayed in a dedicated window in real time, which was managed by the GUI software. Also, the tri-modal imaging system 1 may be configured to support the intermodal registration for the FL and combined US/PA coronal plane (C-plane) images, producing tri-modal images. The GUI software was programmed in Visual C++ on the host system CPU. An example embodiment of a method for tri-modal image registration is described below.

### C. Combined US/PA Subsystem

FIG. 3 shows the overall block diagram of an example embodiment of a combined US/PA subsystem 42′ having a front end or acoustics probe section 42a and a backend signal conditioning/data acquisition backend 42b with optional processing elements. It should be noted that in alternative embodiments there may just be an US subsystem or a PA subsystem. In FIG. 3, the probe section 42b comprises a system trigger controller 100, a platinum resistance bulb connector (PRBC) 102, a relay 104, a high voltage multiplexer (HVM) 106, a transmit beamformer (TxBF) 108, a diode bridge 110, a low-noise amplifier (LNA) 112, an analog-to-digital converter (ADC) 114, a receive beamformer (RxBF) 116 and a digital backend 118. The US/PA subsystem 42′ further comprises the GE DAQ system 7a, a delay generator (DG) 101, the laser source 86b and the optical parametric oscillator (OPO) 86a. The system trigger controller 100 is coupled to the TxBF 108 and the DG 101. The system trigger controller 100 initiates the transmission of US, PA or both US and PA excitation signals for transmission to the portion of the object being imaged. The DG 101 provides certain delays depending on the type of excitation energy that is transmitted.

In the PA mode, for PA excitation, the system trigger controller 100 sends a trigger signal to the DG 101, which creates a series of delays so that the laser source 86b generates a series of laser pulses that are transmitted to the US/PA probe portion 42a for transmission to the object. At the same time, the delay pulses created by the DG 101 are sent to the GE DAQ system to synchronize obtaining data any acoustic energy that is generated by the portion of the object being imaged so that the data can be arranged as acoustic-based image data, which in this case is PA image data. The reception of PA-evoked acoustic energy from the region of interest is received by the ultrasound transducer 80 in the UA/PA probe portion 42a and then transmitted through the diode bridge 110, which acts to rectify the sensed data which is then amplified by the LNA 112 and digitized by the ADC 114. The digitized data may then be beamformed by the digital RxBF 116 to account for the delay between received echo data to determine PA image data at different depths along an A-scan. This may then be repeated to obtain PA image data for a B-scan or for a C-scan.

In US mode, the system trigger controller 100 triggers the TXBF 108 to generate electrical pulses which are transduced to obtain US image data at different depths of the target region 85. The electrical pulses are sent through the HVC 106, the relay 104 and the PRBC 102 to the US probe section 42a where they are transduced into ultrasound signals by the US transducer 80 and sent to the region of interest 85 of the object where US echoes are generated and sent back to the US/PA probe portion 42a. These US echoes are transduced by the US transducer 80 into electrical signals which are then processed similarly as the PA echoes in PA mode.

The example embodiment of the US/PA subsystem 42′ shown in FIG. 3 may be implemented using a commercial US scanner (P6, GE Corp., Gyeonggi-do, Korea), a data acquisition system (GE-DAQ), and a PA excitation laser (which may have a maximum energy density of 31 mJcm⁻², a pulse duration 5-7 nm, a pulse repetition frequency 10 Hz, and a tunable wavelength 680-950 nm, for example).

The US/PA subsystem 42′ may provide stand-alone US and PA imaging modes as well as a combined PA/US imaging mode where the PA/US data are obtained simultaneously in real-time. The raw radio-frequency (RF) US and PA data may be digitized in the analog front-end of the US scanner and transferred to either the internal digital processing unit 116 and 118 (in US mode, for example) where transmit/receive beam forming and back-end processing are conducted, or to the external GE-DAQ system 7a (in PA mode, for example). The reconstructed US images may then be transferred to the host process system 10 through a frame grabber (FG, VGA2USB LR: Epiphan Systems Inc., Ottawa, ON, Canada). The maximum frame rate may be determined by the imaging depth that is used and the number of scan lines: e.g., if it takes 54 μs to acquire a single scan line for an imaging depth of 4 cm, then an US image of 128 scan lines will take 6.9 ms to acquire, giving a maximum frame rate of 145 frames-per-second (fps).

In PA mode, the PA data may be sent to SDRAM buffers for storage in the GE-DAQ unit 7a. The PA data may then be transferred to the host process system via an 8-lane PCI Express bus. Since the PA data corresponds to one frame and may be acquired every laser pulse repetition period, the frame rate is the same as the laser pulse repetition frequency (PRF), i.e., up to 10 fps. PA data acquisition may start whenever the system trigger controller (STC) 100 in the US scanner generates a Q-switch trigger signal, which may also be sent to the low-jitter digital delay generator 101 (DG535; Stanford Research Systems, Inc., Sunnyvale, Calif., USA) to trigger the laser pulse. The DG 101 may provide a signal to accumulate laser energy during the programmed Q-switch delay time and then transmit a FIRE trigger signal to release a laser pulse with the accumulated energy. The maximum light energy density at 700 nm may be about 31 mJ cm⁻² at the output of the optic fiber bundle when the Q-switch delay is 230 μs. The energy density may be controllable with sub-mJcm⁻² precision by adjusting the Q-switch delay and may be set to a maximum of 20 mJ cm⁻² for the near-infrared spectral range to satisfy ANSI exposure limits.²⁷ The laser pulse may be 5-7 ns long and its wavelength may be selected from 680 to 950 nm.

### D. FL Subsystem

FIG. 4 shows the overall block diagram of an example embodiment of the FL subsystem 40′ which comprises a handpiece (e.g. an FL probe portion 40a) and a backend module 40b that includes control electronics and one or more light sources, which are used by the host process system 10 to acquire and process FL and/or white light (WL) image data streams. In some alternative embodiments, there may just be an FL excitation light source for obtaining FL image data.

The FL probe portion 40a comprises a light guide 58 which receives light from one or more light sources and transmits a light beam 126 to the a portion of the object to be imaged. The FL probe portion 40a comprises two optical paths 60 and 62 with light detectors 60a and 62a and zoom optics 60b and 62b, respectively, to obtain stereoscopic FL and/or WL image data. An example embodiment of the cameras 60a and 62a and the zoom optics 60b and 62b were described in more detail with respect to FIGS. 2B-2D. However, in alternative embodiments, there may only be a single optical path (60 or 62) for obtaining FL and/or WL image data. The FL probe portion 40a also comprises a stepper motor 124 for moving certain moveable lens elements in the zoom optics 60b or 62b to obtain image data under different amounts of magnification if desired.

The FL back-end 40b comprises an LED light source 8b having a controller 120, a 400 nm light source 8b and a white light source 8b′. The light sources 8b and 8b′ can be implemented using LEDs, in which case the controller 120 is an LED controller. The controller 120 controls the activation and deactivation of the light sources 8b and 8b′ based on control signals received from a micro-controller 8a. The micro-controller 8a also sends control signals to a stepper zoom motor driver 122 which provides control signals to activate and move the stepper motor 124 by a desired amount to control the amount of zoom provided by the zoom optics 60b and 62b. The micro-controller 8a controls the communication of the FL and/or WL image data that is recorded by the cameras 60a and 60b.

The host process system 10 may communicate through a USB interface 128 with the microcontroller 8a (Arduino Uno Rev3, Ivrea, Italy) to update the status of the light sources 8b and 8b′ and the amount of zoom that is applied when recording FL and/or WL image data. When a zoom position change is requested, the microcontroller 8a may send a pulse sequence (in half-stepping mode, for example) to the stepper motor driver 122 (e.g. a Motor Shield Rev3: Ardino) that moves the stepper motor 124 (e.g. an AM1020: Micromo, Clearwater, Fla., USA) to the new zoom position. When updating the light source status, a command may be sent to the light source (e.g. LED) controller 120 (e.g. a SLC-SA04: Mightex, Toronto, ON, Canada) to adjust the supply current to a selected light emitting diode (LED) 8b or 8b′. The collimated light from both the white (LCS-4000-03-22: Mightex) and violet (LCS-0400-17-22: Mightex) LED modules may be combined using a dichroic beam splitter (LCS-BC25-0410: Mightex) and then may be coupled to a 3 mm liquid light guide 58 (e.g. LLG0338-6: Thorlabs Inc., Newton, N.J., USA) using an adapter (LCS-LGA22-0515: Mightex). The beam splitter and the adapter are not shown in FIG. 4. The two cameras 60a and 62a have by-directional communication with the host process system 10. The data acquisition module 34 and the tri-modal imaging module 36 may directly update the image acquisition rate, gain settings and white balance factors, while also receiving the pixel data stream (i.e. FL and/or WL image data). Processing for 3D reconstruction or image co-registration may also be performed on the host process system via the tri-modal imaging module 36.

### E. Multimodal Image Registration

The combined US/PA images may be obtained in the cross-sectional plane formed by the lateral and axial axes (i.e. the x-z plane in FIG. 2E), e.g. along vertical planes that are perpendicular to the surface of the portion of the object being imaged, while the FL images may be in the C-plane (i.e. the x-y plane in FIG. 2E), e.g., along horizontal planes which may be at the surface of the portion of the object being imaged or at a certain depth below the surface of the portion of the object being imaged. Hence, multi-modal image registration may use multiple cross-sectional US/PA image data from which may be oriented vertically and in parallel with one another to reconstruct a C-plane (i.e. 3D) US/PA image onto which the FL image obtained a certain depth can be overlaid at a corresponding depth in the reconstructed C-plane US/PA image. Mechanical scanning of the tri-modal imaging probe 2 may be used to generate the required 3D US/PA image. In some embodiments, either electromagnetic or optical position tracking sensors may be used for registration during hand-held operation.

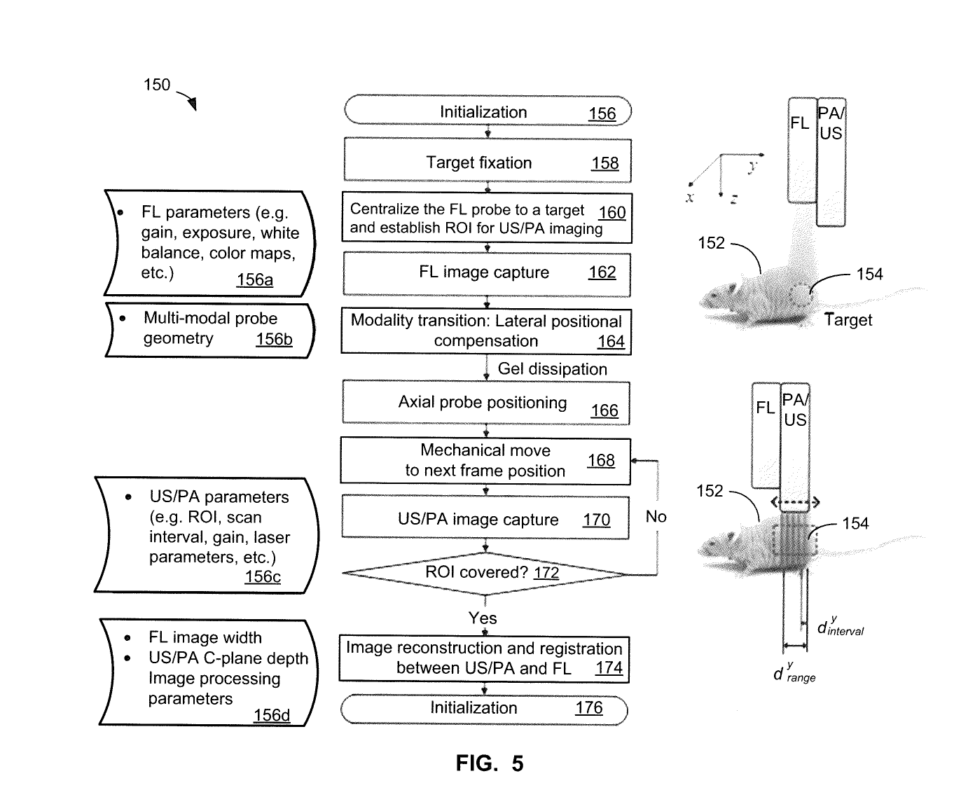

A flow chart showing an example embodiment of a method 150 for data acquisition, image reconstruction and image registration for tri-modal imaging is shown in FIG. 5 (in which the parameters the and dᵢₙₜₑᵣᵥₐₗ^{y} and d_{range}^{y} are the interval and overall range for the 3D mechanical scanning of the combined US/PA imaging, respectively).

In this example embodiment, the method 150 begins with initializing various parameters at act 156. The initialization act 156 depends on the type of imaging that will be performed. For FL imaging, the initialization includes act 156a where values may be set for various FL parameters such as, but not limited to, gain, exposure, white balance, and color maps, for example. Initialization may also include obtaining values for the multimodal probe geometry at 156b. For PA and/or US imaging, initialization includes act 156c where various parameters are set for PA/US parameters such as, but not limited to, ROI, scan interval, gain, and laser parameters, for example. Finally, initialization may also include act 156d where various image processing parameters are obtained to allow for the reconstruction of FL, PA and/or US images as well as the co-registration and merging of FL image data with at least one of PA image data and US image data. The image processing parameters may include, but are not limited to, FL image width, PA/US C-plane depth as well as other image processing parameters, for example. The various parameters may be obtained from one or more databases or may be provided by a user of the imaging system.

The method 150 then proceeds to act 158 at which point a target is fixated, where the target 154 is the portion of the object 152 that is to be imaged. At 160, the FL portion of the probe 1 is centered on the target and a Region of Interest (ROI) 154 over which FL image data will be acquired is determined. The FL image data is then acquired for the ROI 154 at act 162 while the FL portion of the probe is centered on the target as shown in the figure inset at the top right of the flowchart.

The method 150 may then comprise acquiring 3D US/PA imaging data at acts 164 to 172. Act 164 includes properly positioning the US/PA portion of the probe to obtain US image data and/or PA image data for the ROI 154. Gel is then applied to the US/PA probe portion and the US/PA probe portion is axially positioned with respect to the ROI at act 166. The position of the US/PA probe portion may then be adjusted along the y dimension, for example, by mechanically translating the probe 1 along the elevation (y) direction using the mechanical scan system 5 at act 168. The US image data and/or PA image data is then captured at act 170. Acts 168 and 170 are repeated so that 2D US/PA imaging may be performed sequentially by moving the probe in increments of dᵢₙₜₑᵣᵥₐₗ^{y}, as shown in the figure inset at the bottom right of the flowchart, until the whole ROI is covered. For a total scanning distance of d_{range}^{y}, the number of the cross-sectional US/PA data frames may then be rounded (e.g. round (d_{range}^{y}/dᵢₙₜₑᵣᵥₐₗ^{y})+1, where round () denotes rounding to the nearest integer). For optimal registration, the FL image may be centered at the midpoint of d_{range}^{y}, so that the initial US/PA scanning position may be determined by considering the elevation distance, d_{FL-PAUS}^{y}, between the centers of the FL and the US/PA axes. After acquiring the 3D US/PA data, both the PA and US C-plane images may be reconstructed.

At act 174, image reconstruction of the FL image and the US image and/or PA image occurs. Act 174 may optionally include co-registering the FL image with the US images, PA images or the US and PA images that have image information corresponding to the plane of the target from which the FL image was obtained as described previously.

In at least some embodiments, the range of depths of interest (DOI) for the C-plane reconstruction may be selected in a GUI provided by the software, such as the tri-modal imaging module 36. The corresponding C-plane images may then be used to produce the maximum-intensity projected C-plane images (MAP).

In at least some embodiments, the PA and US C-plane images may be selectively alpha-blended (combining a translucent foreground color with a background color, producing a new blended color) to the center of the FL image. This may involve scaling the US/PA C-plane images to have the same pixel size as the FL image, using a scaling factor, an example of which is shown in equation 2:

$${R}_{resize } = \frac{Δ {d}_{FL}}{Δ {d}_{US / PA }} , \qquad ( 2 )$$

where Δd_{FL} and Δd_{US/PA} are the pixel sizes in the FL and US/PA images, respectively. The pixel size Δd_{US/PA} may be fixed at 0.1 mm (e.g., 128×400 pixels for 12.8 mm aperture size and 40 mm imaging depth). By contrast, the pixel size Δd_{FL} may vary with the axial distance between the tri-modal imaging probe face and the tissue surface. Thus, for example, with a fluorescence field-of-view of 120 mm by 90 mm, the pixel size is 0.185 mm for a 648×486 image, so that the US/PA images may be enlarged by a factor of 1.85 times for tri-modal image registration.

In an alternative embodiment of the method 150, only PA image data is obtained for co-registration and combination with the FL image data in a similar fashion as was described for the US/PA image data in method 150. In a further alternative embodiment, only US image data is obtained for co-registration and combination with the FL image data in a similar fashion as was described for the US/PA image data in method 150.

### F. Performance Testing

For high quality PA imaging, the laser energy may be uniformly distributed over the ROI. This can be evaluated by measuring the laser energy density along the lateral and the elevation directions at a given depth. For this measurement, the US/PA subsection of the tri-modal imaging probe may be moved on a mechanical arm along the 3 directions above a fixed spectral photosensor (AvaSpec-2048x16 Spectrometer: Avantes BV, Apeldoorn, The Netherlands). The energy distribution in the axial direction was first determined by translating the probe depth from of 0 to 14 mm in 1 mm increments. The photon counts in the lateral and the elevation directions were measured at the geometric focal depth of 7 mm of the US transducer 80 over 16 mm and 10 mm in increments of 2 mm and 1 mm, respectively. The photosensor output was sampled 100 times and the procedure was repeated 6 times.

The performance of the custom-made US linear transducer array was also evaluated by a pulse-echo test²⁸⁻²⁹ in which the center frequency, fractional bandwidth and loop gain were measured using a stainless steel reflector immersed in distilled water at the 7 mm focal depth. A commercial pulse-receiver (5900PR-102: Olympus, Corp., Tokyo, Japan) was used to excite the transducer and receive the echo, which was then recorded by a digital oscilloscope (TDS3052C: Tektronix, Ltd., Puerto Rico, USA) at 500 MHz. The performance metrics were determined using MATLAB software (Mathworks, Inc., Natick, Mass., USA).

The spatial resolution of the US/PA system was determined by immersing a 0.3 mm diameter graphite rod in water at the geometric focal depth. The PA image was acquired and the axial and lateral resolutions were determined. In practice, the spatial resolution may generally be determined by the center frequency, the fractional bandwidth and the aperture size of the US transducer array, but may also be affected by the reconstruction method. In addition, the signal processing time used to reconstruct a PA image was measured using the QueryPerformanceCounter function of C++ that is capable of measuring processing times up to 1 ms.

For the FL subsystem described herein, the spatial resolution was first determined by imaging a 10 μm diameter pinhole at 650 nm and measuring the full-width-at-half-maximum (FWHM) of the digital image. To test the response sensitivity and linearity, glass vials containing different concentrations of protoporphyrin IX (Sigma, MO, USA) were imaged at 10 fps. The PpIX is a common fluorophore for clinical diagnostics and image-guided surgery.¹⁹ Under inhalation of general anesthesia, in vivo imaging was then demonstrated in a mouse model bearing a 5 mm diameter subcutaneous breast cancer xenograft, by intratumorally injecting 50 μl of 77 μM PpIX and acquiring images at 10 fps within a few minutes. The fluorophore volume and concentration were selected to be high enough to be well within the sensitivity response range of the FL system, and are typical of those used in previous studies. All procedures were carried out with institutional ethics approval (University Health Network, Toronto, Canada). All images were captured at an optimal working distance of 12 cm.

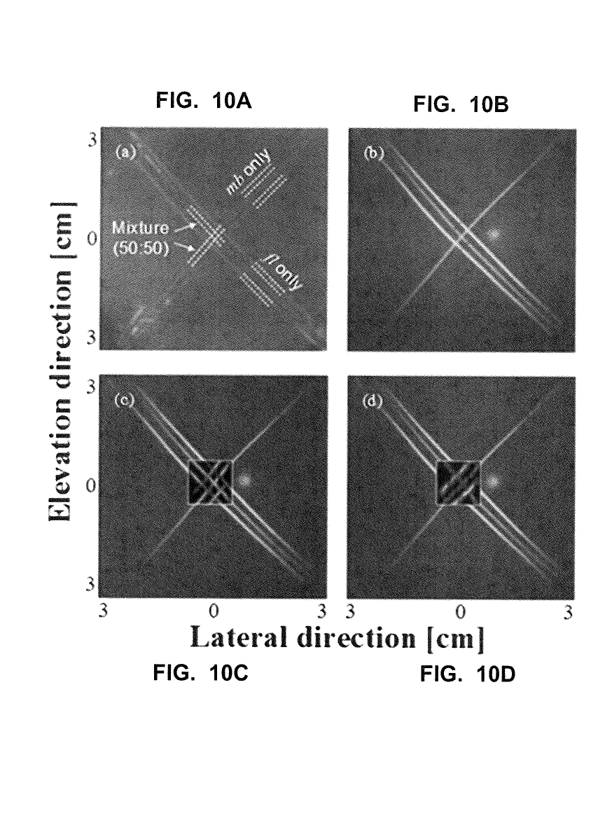

To test the tri-modal imaging performance, a phantom experiment was first performed to show the capability of the image registration using the tri-modal imaging probe. The phantom comprised an acoustic gel pad (Aquaflex: Parkers Lab, Inc., Far-field, NJ, USA) with embedded flexible plastic tubing having 1/16 and 3/16 inch inner and outer diameters (Tygon®-2075, Z279803: Saint-Gobain Performance Plastics, La Defense, Courbevoie, France) placed in a crossed 2×3 configuration as shown in FIG. 10A. The distance between tubes was approximately 2.5 mm. The center tubes were filled with a mixture of methylene blue and fluorescein (M9140 and F6377: Sigma-Aldrich Co., St. Louis, Mo., USA) of equal volume (i.e., 100 μL each), giving molar concentrations of 1.56 mM and 1.32 μM, respectively. The side tubes contained methylene blue only in one set of 3 tubes and fluorescein only in the crossed set, with concentrations of 2.66 mM and 2.66 μM, respectively.

In vivo experiments were conducted under the approval of the Institutional Review Board (IRB) at Sogang University and Seoul National University Bundang Hospital. A 2-week old B16F10 mouse was injected subcutaneously with B16 melanoma cells and incubated for 2 weeks for tumor growth, at which time 200 μL of the mixture was injected into the tumor at 10 min prior to imaging under gas inhalation anesthesia. The purpose of the phantom and in vivo experiments was to demonstrate the tri-modal functionality of the tri-modal imaging probe, rather than to determine the imaging sensitivity limits of each modality.

For both the phantom and in vivo experiments, the tri-modal imaging probe was first positioned at 120 mm from the target and the FL image was acquired. PA and US images were then acquired after repositioning the tri-modal imaging probe to 5 mm from the target and scanning over the range, d_{range}^{y}, of 12 mm (phantom) and 10 mm (tumor) at dᵢₙₜₑᵣᵥₐₗ^{y}=1 mm increments. A MAP image was then generated from the 3D US/PA C-plane images, with DOIs of 5-10 mm. The PA laser exposure was maintained at <10 mJcm⁻² at the front of the US/PA probe, as verified by averaging 500 sequential pulses with a calibrated energy meter and sensor (MAESTRO and QE25, Gentec Electro-Optics Inc., Quebec, QC, Canada).

### G. Results

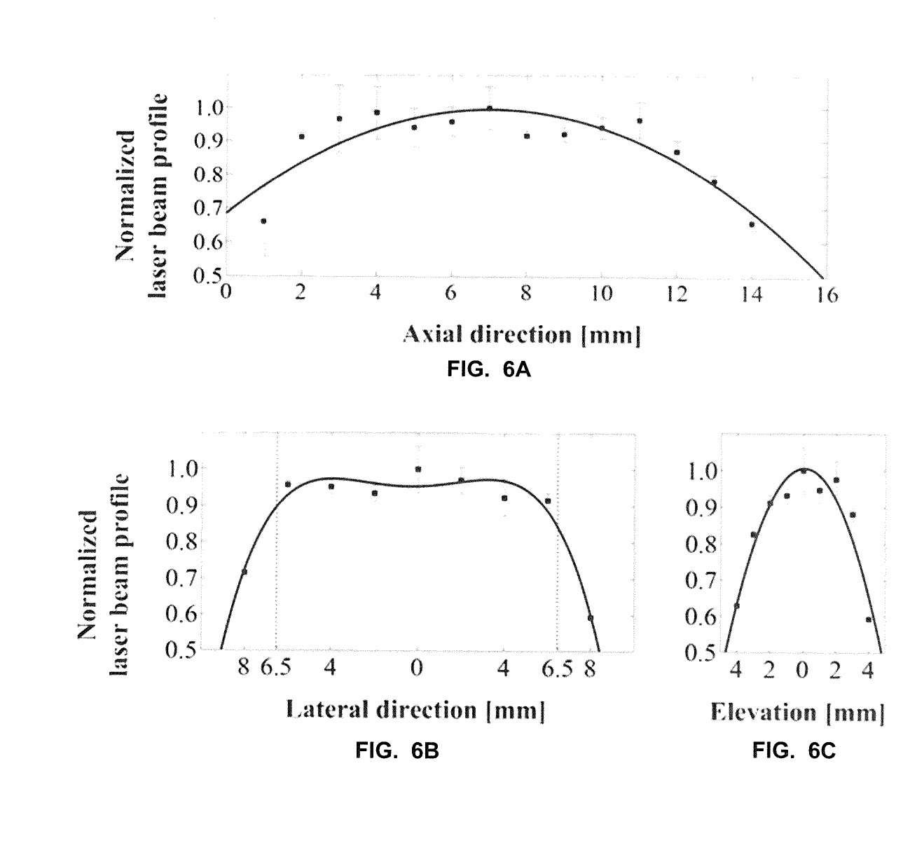

FIGS. 6A-6C show the PA light beam profiles in which the solid lines indicate laser beam profile curves fitted by quadratic and 4^{th} degree polynomials. FIG. 6A demonstrates that the PA laser beam profile in the axial direction may be optimal over the depth range 2-12 mm which may indicate that the PA signal may be maximized at the geometrical acoustic focal depth of the transducer (7 mm). Also, as shown in FIG. 6B, over 90% of the optical energy in the lateral direction was uniformly distributed within the imaging width (i.e., 12.8 mm) at the acoustic focal depth, but was diffused along the elevation direction. In FIG. 6B, the dotted line in the lateral beam profile indicates the image width of the US/PA submodule.

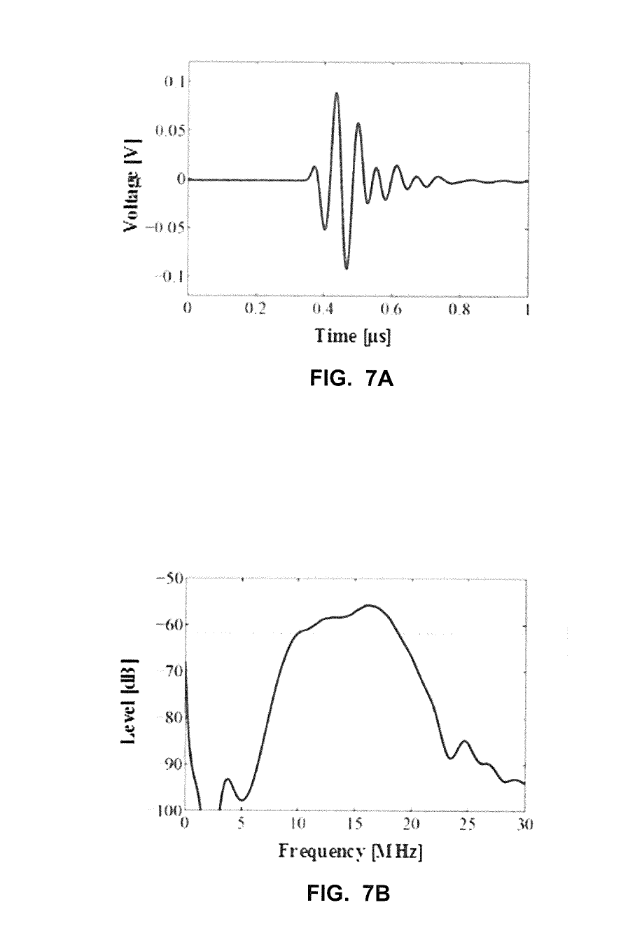

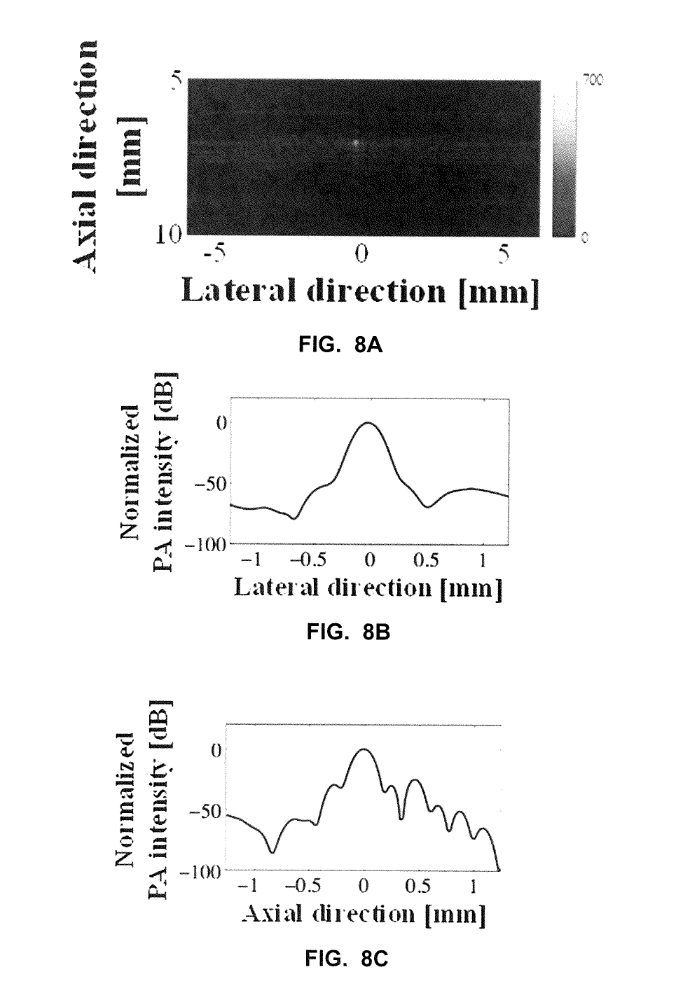

The pulse-echo response and frequency spectrum of the US array are shown in FIGS. 7A-7B. The center frequency, fractional bandwidth and loop gain were 14.21 MHz, 65.9% and 55.92 dB, respectively. Also, the spatial resolution was quantitatively evaluated from the PA image as shown in FIGS. 8A-8C. The −3 dB spatial resolution was 124 μm and 117 μm in the lateral and axial directions, respectively. Note that the −3 dB spatial resolution corresponds to the −6 dB spatial resolution of US images. In addition, the total processing time, defined as the time from data acquisition to image display, was measured multiple times as shown in Table 4. As described above, signal processing for the reconstruction consumed 9.9 ms per cross-sectional image, which was only 10% of the maximum available for the combined US/PA subsystem. The frame rate for combined US/PA imaging was determined by the PRF of the OPO system, i.e., 10 Hz. Transfer of the raw photoacoustic RF data may be on average 55.6 ms due to the limited data transfer throughput of the PCI Express 2.0 bus.

TABLE 4
Processing time of CUDA processing in PA image reconstruction
with 4 cm imaging depth: i.e., 1024 analog-to-digital converted samples per
channel, 40 MHz sampling frequency, 1540 m/sec speed of sound in tissue.

| | | Processing |
|---|---|---|
| Processing stages | | time [ms] |
| Q-switch delay for determining laser energy | | 0.2-0.4* |
| Data transfer | From GE P6 system to GE-DAQ | 0.6 |
| | From GE-DAQ to GPU memory | 55 |
| Image reconstruction | Receive beamforming | 6 |
| | Back-end processing | 2.6 |
| | Image display | 1.3 |

*can be varied corresponding to the desired laser energy

The FWHM of the fluorescence point source response function was measured as 120 μm at 1× zoom. The corresponding resolution of the optical chain, based on the determined modulation transfer function, was less than approximately 10 μm. Hence, the resolution may be limited by the detector. In examining the raw signal from the CMOS camera(s), the image of the pinhole was essentially 1 pixel in size (33 μm) but this was increased to about 3 pixels (100 μm) in the full-color image, indicating that the Bayer filter may be responsible for the loss of resolution. Nevertheless, given that in vivo fluorescence imaging in tissue is significantly degraded by light scattering in tissue, a resolution of 120 μm is adequate for the intended applications.

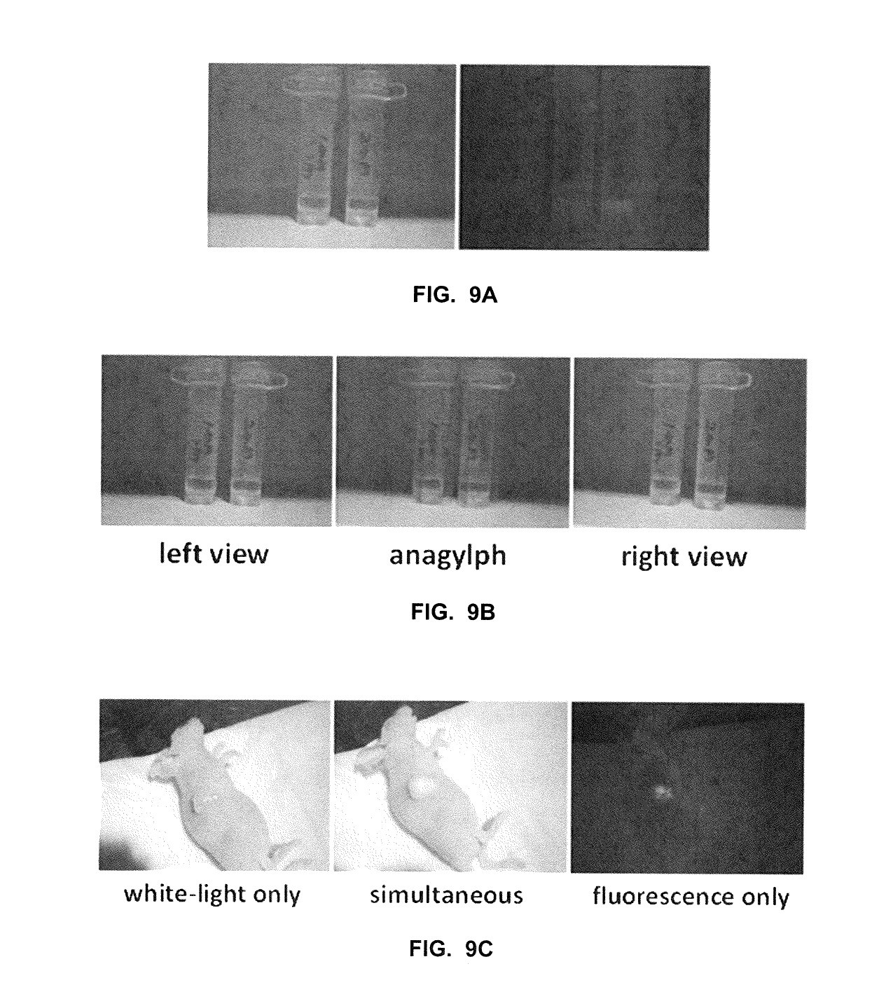

FIG. 9A shows the FL images of two different concentrations of PpIX solution. After background subtraction, the measured ratio of the integrated fluorescence intensity in Tube 2 to that in Tube 1 was 2.2, which is consistent with the 2:1 ratio of PpIX concentration. FIG. 9B demonstrates the stereoscopic capability of the tri-modal imaging probe 1, displayed as a red-cyan anaglyph image (in the center panel). FIG. 9C shows images of the tumor-bearing mouse in reflectance (white-light only), reflectance plus fluorescence (under simultaneous white-light and blue-light illumination), and fluorescence only (from the left panel to the right panel, respectively). The PpIX fluorescence in the tumor is clearly seen, even through the overlying skin and under normal ambient lighting. The estimated signal-to-noise ratio in the fluorescence image was about 4:1 in the red channel for the specific concentration of PpIX used here. Note that there is no marked tissue autofluorescence background visible in these images outside the PpIX (red) fluorescent tumor region. However, the fluorescence images also show a faint blue background due to leakage through the cut-on filter (out-of-band optical density of 4) of some excitation light. This may be eliminated in some embodiments, but in fact has utility since it may also enable visualization of the overall tissue context/anatomy when working in FL mode and is commonly reported in, e.g. fluorescence image-guided surgery using PpIX.¹⁹

Tri-modal phantom images are presented in FIGS. 10A-10D. The white-light image (FIG. 10A) shows the configuration of the fluorophore-filled tubes. The FL image (FIG. 10B) shows only the fluorescein contribution due to a poor fluorescence yield of methylene blue³⁰, and so serves as a negative control. The fluorescein concentration dependence of the fluorescence intensity is also clearly seen. FIGS. 100 and 10D demonstrate the capability for tri-modal image fusion, by overlaying either the US or PA C-plane images on the FL image. Since the reflection of the acoustic wave occurs regardless of the tube contents, the US C-plane image depicts the full configuration of the tubes (i.e. the “structural” image), while the PA images reveals only the tubes containing methylene blue since the fluorescein does not have significant absorption at the photoacoustic wavelength of 700 nm. As for FL imaging, the pixel intensity depended on the dye concentration. There may be some discontinuities in the PA image due to blocking of acoustic wave propagation toward the US transducer by the overlapping tubes.

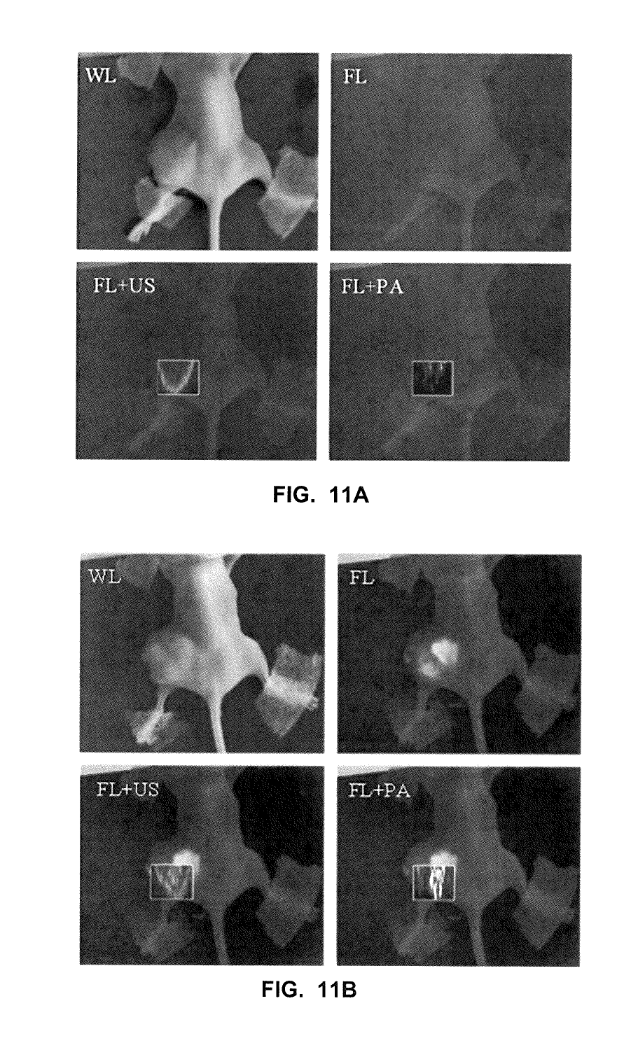

FIGS. 11A and 11B show a series of in vivo tumor images before and after the injection of an equal mixture of methylene blue and fluorescein, respectively. The pre-injected tumor shows minimal fluorescence, while the PA image (700 nm) shows the expected intrinsic signal from the tumor vasculature due to the light absorption by blood (hemoglobin).¹³ Following dye injection, the fluorescein fluorescence is clearly visible throughout the tumor mass, while the PA signal (methylene blue) is both more uniform and more intense than the intrinsic signal.

### H. Discussion

The multi-modal imaging system described herein that integrates US, PA and FL imaging into a single device has been shown to be suitable for in vivo imaging in animal models and is compact and lightweight enough for handheld use. In at least one embodiment, this system may use a de novo design for a compact handpiece and incorporation of a PA pulsed-laser light source and light sources and cameras for stereo-zoom FL (and optionally white-light) imaging. Initial characterization indicates that all imaging modes perform separately well enough for in vivo imaging under realistic conditions and that the tri-modal image integration works with no signal degradation due to combining these different imaging modalities. Tests herein have shown the feasibility of a first approach to co-register the US/PA and FL image data, which are obtained in orthogonal planes. Also, in example embodiments where the US/PA and FL functionality is achieved in the tri-modal imaging probe using independent hardware, little compromise is needed beyond that involved when using each imaging technique alone.

The tri-modal imaging probe, methodology and system in accordance with the teachings herein may be used in a wide variety of preclinical and clinical applications.

For example, at least some embodiments described herein may be used to support standard approaches to preclinical and clinical imaging with each of the separate US, PA and FL modalities as may be possible using other single-modality instruments, since the tri-modal imaging probe can be separated into individual subsections or can be operated while using only one imaging modality.

As another example, at least some of the embodiments described herein may be used to provide more comprehensive information using tri-modal imaging, as illustrated by the examples in FIGS. 10A-10D and 11A-11B. This may expedite imaging of early indicators of diseases (e.g., abnormal morphology such as microcalcifications, neovasculature or altered oxygen saturation) or therapeutic responses, which are generally challenging to monitor in heterogeneous biological tissue³¹⁻³⁴.

As another example, while the penetration depth of FL imaging may be limited compared to that of US and PA imaging (see Table 1 for example), when at least some of the embodiments described herein are used in certain applications, FL imaging along with US and/or PA imaging may provide additional morphological or functional context. For example, fluorescence tomography anatomic information from CT or MRI is often used to correct the fluorescence images for the effects of light attenuation by the tissues.⁶ Here, the fluorescence is primarily surface or near-surface, so that such attenuation correction is not relevant in terms of image reconstruction. However, if quantitative fluorescence imaging techniques are incorporated into future versions of the tri-modal imaging system, then attenuation correction may be applied based on the morphology and vascular distribution from the US/PA images and using reference tissue optical properties.

As another example, at least some of the embodiments described herein may be used for guiding tumor resection which may include first generating the US/PA images to delineate the anatomy/microvascularity to guide tumor debulking, followed by FL imaging or interleaved with FL imaging (e.g. alternating between US/PA imaging and FL imaging) to identify tumor margins, thereby ensuring maximal safe resection³⁵.

Some modifications may also be made to at least one of the embodiments described herein to improve performance. Firstly, the maximal temporal resolution of the prototype system described herein is currently about 10 fps, which is mainly determined by the 10 Hz PRF of the laser. For more efficient real-time diagnosis, the frame rate may be increased to be larger than 20 fps, while maintaining the laser energy up to 20 mJ/cm².

Another improvement that may be done to at least one of the embodiments described herein may be to reduce the total processing time for PA image reconstruction. Since the data transfer rate from the GE-DAQ system to the host process system 10 is the main time consumer (i.e., 55.6 ms), PA image reconstruction may be improved by changing the data transfer protocol from PCI Express 2.0 to PCI Express 3.0 and to further optimize the GE-DAQ system for reduction of internal latency.

Another improvement that may be made to at least one of the embodiments described herein may be to increase the field of view of the US/PA subsection of the tri-modal imaging probe to support various clinical applications. This may involve changing the configuration of the US/PA subsection, including changing the US transducer aperture size and/or operating frequency of the US transducer, the size of the optical fiber bundle and/or the optical/acoustical focal depths. These changes may be done to optimize the use of the tri-modal imaging probe for use in a particular application.

Another improvement that may be made to at least one of the embodiments described herein may be to further improve the optics in the FL subsection of the tri-modal imaging probe to improve performance. For example, while the prototype embodiment described herein allows for high-quality FL imaging in vivo, the long zoom switch time (currently 5 s) may be improved by swapping the stepper motor and mechanical drive components.

Furthermore, while the FL mode has been implemented in some of the embodiments described herein with specific single excitation and detection wavelength bands, in other embodiments user-selectable excitation wavelength may be incorporated by including multiple LED sources that can produce light at different wavelengths and using either optical or electronic switching. However, including the ability to switch the detection wavelength during use in at least one of the embodiments described herein may require incorporating spectral filtering into the tri-modal imaging probe before the light reaches the detector array, which may necessitate some reconfiguration of the layout. Pre-section of the detection wavelength band prior to use may be achieved by using a different long-pass or band-pass filter element.

At least one of the embodiments described herein may be modified to refine the co-registration and display methods to address specific clinical needs. For example, incorporating electromagnetic or optical tracking onto the tri-modal imaging probe may also enable co-registration with radiological (e.g. CT or MR) volumetric images. For preclinical (e.g. small animal) applications, having the tri-modal imaging probe itself or the animal mounted on an accurate translation stage, an example of which is illustrated in FIG. 1A, will facilitate accurate co-registration and longitudinal monitoring of, for example, tumor development and therapeutic response.

In another aspect, in at least one embodiment there is a sensor that is configured to detect at least two signals responsive to illumination of the ROI of the object, each signal being indicative of at least one of optical reflectance, endogenous fluorescence, exogenous fluorescence, optical absorbance, photoacoustic absorbance and ultrasonic energy in the illuminated portion (i.e. ROI) of the object.

In another aspect, in at least one embodiment there is at least one light source that is coupled to the housing of the multi-modality imaging probe and is movably positionable with respect to the housing to vary an illumination of at least one wavelength of light and an illumination angle and distance to an object's ROI for imaging; and a controller that is coupled to the light source and configured to modulate the light source for obtaining PA/WL/FL 2D/3D image data of the ROI of the object.

In another aspect, in at least one embodiment there is a processor for processing detected multimodal image data and outputting a representation of a ROI of an object from which the image data was obtained, wherein the processor may be configured to spatially co-register the multimodal image data relative to at least one of object topography, object anatomy, object area, object heterogeneity and composition, object depth, object volume, object margins, and necrotic tissue; a user interface configured to allow a user to interact with a multi-modal imaging probe and associated subsystems; and a display for displaying the multimodal representation of the ROI. The representation may be indicative of at least one of a presence of at least one of healthy tissue, vasculature, bone, and tumor or diseased tissue, at the ROI of the object; at least one of a location, a quantity, a distribution, and an extent of at least one of healthy tissue, vasculature, bone, and tumor or diseased tissue when present in the ROI of the object; and at least one of a presence, a location, a distribution, and an extent of at least one of cells, molecules, and fluids indicative of disease in the ROI of the object.

It should be noted that various embodiments of systems, processes and devices have been described herein by way of example only. It is not intended that the applicant's teachings be limited to such embodiments. On the contrary, the applicant's teachings described and illustrated herein may encompass various alternatives, modifications, and equivalents, without departing from the spirit and scope of the embodiments described herein, which is limited only by the appended claims, which should be given the broadest interpretation consistent with the description as a whole.

### REFERENCES

[1] Hicks R., Lau E., and Binns D.., Biomed. Imaging Interv. J. 3, e49 (2007).
[2] Marti-Bonmatí L., Sopena R., Bartumeus P., and Sopena P., Contrast Media Mol. Imag., 5, 180 (2010).
[3] Solomon, M., Nothdruft, R. E., Akers, W., Edwards, W. B., Liang, K., Xu, B., Suddlow G. P., Deghani H., Tai Y. C., Eggebrecht A. T., Achilefu, S., and Culver J. P., J. Nucl. Med. 54, 639 (2013).
[4] Papathanassiou D., Bruna-Muraille C., Liehn J. C., Nguyen T. D., Curé H., Crit. Rev. Oncol. Hematol., 72, 239 (2009).
[5] Torigian D. A., Zaidi H., Kwee T. C., Saboury B., Udupa J. K., Cho Z. H., Alavi A., Radiology, 267, 26 (2013).
[6] Mohajerani P., Hipp A., Willner M., Marschner M., Trajkovic-Arsic M., Ma X., Burton N. C., Klemm U., Radrich K., Ermolayev V., Tzoumas S., Siveke J. T., Bech M. F., and Ntziachristos V., IEEE Trans Med Imaging, 33, 1434 (2014).
[7] Li B. H., Leung A. S., Soong A., Munding C. E., Lee H., Thind A. S., Munce N. R., Wright G. A., Rowsell C. H., Yang V. X., Strauss B. H., Foster F. S., Courtney B. K., Catheter Cardiovasc Interv., 81, 494 (2013).
[8] Georgakoudi I., Sheets E. E., Müller M. G., Backman V., Crum C. P., Badizadegan K., Dasari R. R., Feld M. S., Am. J. Obstet. Gynecol., 18, 374 (2002).
[9] Lorenser D., Quirk B. C., Auger M., Madore W. J., Kirk R. W., Godbout N., Sampson D. D., Boudoux C. and McLaughlin R. A., Opt Lett., 38, 266 (2013).
[10] Xi L., Duan C., Xie H., Jiang H., Appl Opt. 52, 1928 (2013).
[11] Dame C., Lu Y., Sevick-Muraca E. M., Phys Med Biol. 59, R1 (2014).
[12] Lin Y., Barber W. C., Iwanczyk J. S., Roeck W., Nalcioglu O. and Gulsen G., Opt. Express, 18, 7835 (2010).
[13] Wang L. V., and Hu S., Science, 335, 1458 (2012).
[14] Zackrisson S., van de Ven S. M. and Gambhir S. S., Cancer Res, 744, 979 (2014).
[15] Mehrmohammadi M., Yoon S. J., Yeager D., Emelianov S. Y., Curr Mol Imaging, 2, 89 (2013).
[17] Goetz M. and Wang T. D., Gastroenterol, 138, 828 (2010).
[18] Zhang H., Uselman R. R. and Yee D., Expert Opin Med Diagn. 3, 241 (2011).
[19] Valdes P. A., Jacobs V. L., Wilson B. C., Leblond F., Roberts D. W. and Paulsen K. D., Opt. Lett. 38, 2786 (2013).
[21] Kim J. S., Kim Y. H., Kim J. H., Kang K. W., Tae E. L., Youn H., Kim D., Kim S. K., Kwon J. T., Cho M. H., Lee Y. S., Jeong J. M., Chung J. K. and Lee D. S., Nanomed., 72, 219 (2012).
[23] Huynh E., Jin C. S., Wilson B. C., and Zheng G., Bioconj Chem., 25, 796 (2014).
[24] Yu P., Ji Y., Neumann N., Lee S. G., Luo H., and Es-Souni M., IEEE Trans. Ultrason. Ferroelectr. Freq. Control, 59, 1983 (2012).
[25] Lee Y., Lee W. Y., Lim C. E., Chang J. H., Song T. K., and Yoo Y., IEEE Trans. Ultrason., Ferroelect. Freq. Contr. 59, 573 (2012).
[26] Chang J. H., Sun L., Yen J. T., and Shung K. K., IEEE Trans. Ultrason. Ferroelectr. Freq. Control, 56, 1490 (2009).
[27] American National Standards Institute (ANSI), Standard Z136.1-2007 (Laser Institute of America, Orlando, Fla., 2007).
[28] Zhuang B., Shamdasani V., Sikdar S., Managuli R., and Kim Y., IEEE Trans. Inf. Technol. Biomed., 13, 571 (2009).
[29] Erikson K. R., IEEE Trans. Sonics Ultrason. 26, 453 (1979).
[30] Gundy S., der Putten W. V., Shearer A., Ryder A. G., and Ball M., in Proc. SPIE 4432, 299 (2001).
[31] J. Kang, E. K. Kim, G. R. Kim, C. Yoon, T. K. Song, and J. H. Chang, J. Biophotonics, DOI 10.1002/jbio.201300100, 1-10 (2013).
[32] G. R. Kim, J. Kang, J. Y. Kwak, J. H. Chang, S. I. Kim, H. J. Kim, and E. K. Kim, PLoS ONE, 9, e105878 (2014).
[33] J. Kang, S.-W. Kang, H. J. Kwon, J. Yoo, S. Lee, J. H. Chang, E. K. Kim, T. K. Song, W. Y. Chung, and J. Y. Kwak, PLoS ONE, 9, e113358 (2014).
[34] R. I. Siphanto, K. K. Thumma, R. G. M. Kolkman, T. G. van Leeuwen, F. F. M. de Mul, J. W. van Neck, L. N. A. van Adrichem, and W. Steenbergen, Opt. Express, 13, 89 (2005).
[35] K. R. Bhushan, P. Misra, F. Liu, S. Mathur, R. E. Lenkinski, and J. V. Frangioni, J. Am. Chem. Soc., 130, 17648 (2008).

1. An imaging probe for obtaining image data of a region of interest (ROI) for a portion of an object using Fluorescence (FL) imaging and at least one acoustics-based imaging modality, wherein the imaging probe comprises:
a Fluorescent (FL) probe portion for obtaining FL image data of the ROI along a first plane at a certain depth of the object, the FL probe portion having a first longitudinal axis and being configured to deliver FL excitation energy from a first end of the imaging probe; and
an acoustics probe portion for obtaining acoustics-based image data of the ROI along a second plane at various depths of the object, the acoustics probe portion being adjacent to the FL probe portion and having a second longitudinal axis that is parallel, co-planar and offset with respect to the first longitudinal axis of the FL probe portion and being configured to deliver excitation energy from the first end to evoke acoustic echoes from the ROI, wherein the FL image data and the acoustics-based image data can be obtained separately or synchronously with one another.

2. The imaging probe of claim 1, wherein the acoustics probe portion comprises an Ultrasound (US) transducer for delivering acoustic energy as the excitation energy and receiving the acoustic echoes for obtaining US image data as the acoustics-based image data.

3. The imaging probe of claim 1, wherein the acoustics probe portion comprises a light guide to deliver light energy as the excitation energy and an US transducer for receiving acoustic echoes generated by a Photoacoustic (PA) response from the ROI and the acoustics-based image data comprises PA image data, and wherein the acoustics probe portion is configured to deliver light energy, acoustic energy or light energy and acoustic energy as the excitation energy and to obtain PA image data, US image data or PA/US image data, respectively.

4. The imaging probe of claim 3, wherein the US transducer emits acoustic excitation energy at an acoustic focal depth that corresponds to the depth of the ROI and the light guide is bifurcated and comprises two output apertures disposed on either side of the US transducer that are angled to output two light beams that overlap at a PA focal depth that is similar to the acoustic focal depth taking into account dispersion of the light beams in the object.

5. The imaging probe of claim 1, wherein the FL probe portion comprises:
a light guide for delivering FL excitation light energy to the ROI, the light guide having a longitudinal axis that is parallel with a longitudinal axis of elements in the acoustics probe portion that deliver excitation energy to the ROI;
a first optical path that comprises a light detector and zoom optics for obtaining the FL image data from the ROI at a desired magnification, the zoom optics being coupled to the light detector and having moveable lens elements; and
a motor for actuating the moveable lens elements in the zoom optics for achieving the desired magnification.

6. The imaging probe of claim 5, wherein the FL probe portion further comprises a second optical path that comprises a second light detector and second zoom optics for obtaining additional FL image data from the ROI at the desired magnification to provide stereoscopic FL image data, the second zoom optics being coupled to the second light detector and having moveable lens elements that are controlled by the motor.

7. The imaging probe of claim 1, wherein the acoustics probe portion has an end portion that is adapted to contact a surface of the object during acoustics-based imaging and the FL probe portion has an end-portion with a stand-off relative to the end portion of the acoustic probe portion so as not to contact the surface of the object during FL imaging.

8. The imaging probe of claim 1, wherein the probe is portable and handheld.

9. A system for obtaining image data of a region of interest (ROI) for a portion of an object using Fluorescence (FL) imaging and at least one acoustics-based imaging modality, the system comprising:
a multi-modal imaging probe comprising an FL probe portion for obtaining FL image data of the ROI and an acoustics probe portion for obtaining acoustics-based image data of the ROI, the FL image data being obtained along a first plane at a first depth of ROI of the object and the acoustics-based image data being obtained along a plurality of second planes along various depths of the object about the ROI, the first and second planes having an angular relationship; and
a processing unit for controlling the system to operate in various imaging modes of operation including a combined FL and acoustics-based imaging mode wherein the FL image data and the acoustics-based image data and portions of the acoustics-based image data obtained along the different second planes at the first depth are combined into multimodal image data by performing intermodal image registration.

10. The system of claim 9, wherein the acoustics probe portion is configured to deliver light energy, acoustic energy or light energy and acoustic energy as excitation energy to obtain Photoacoustic (PA) image data, Ultrasound (US) image data or PA/US image data, respectively.

11. The system of claim 9, wherein the system further comprises a mechanical scan system to move the probe relative to the object to obtain acoustics-based image data along the different second planes of the ROI or from different angles around the ROI.

12. The system of claim 10, wherein the processing unit is configured to generate at least one of reconstructed 2 dimensional (2D) and 3 dimensional (3D) PA and FL image data, combined US/PA image data and combined FL/PA/US image data.

13. The system of claim 10, wherein the processing unit is configured to generate the multimodal image data by performing the intermodal image registration of the FL image data and one of US coronal plane (C-plane) image data, PA C-plane image data or combined US/PA C-plane image data.

14. The system of claim 13, wherein the combined US/PA C-plane image data are in multiple cross-sectional planes formed by lateral and axial axes, the FL image data are in a second C-plane at a given depth and the intermodal registration is based on using the multiple cross-sectional US/PA C-plane image data at the given depth to construct a C-plane US/PA image and onto which the FL image data is overlaid.

15. A method for obtaining image data of a region of interest (ROI) for a portion of an object, the method comprising:
positioning a multi-modal imaging probe with respect to the ROI;
providing excitation light energy along a first longitudinal axis to the ROI to obtain Fluorescent (FL) image data from the ROI at the target of the object using the FL sensor, the FL image data obtained along a plane at a first depth of the ROI;
providing excitation energy along a second longitudinal axis to the ROI to obtain acoustics-based image data from the ROI along a second plane for a plurality of depths of the ROI, the second longitudinal axis being collinear, coplanar and offset with the first longitudinal axis and the acoustics-based image data comprising Ultrasound (US) image data, Photoacoustic (PA) image data or US image data and PA image data; and
combining the FL image data and the acoustics-based image data to generate multi-modal image data by performing intermodal image registration based on an orientation of the first and second planes and the offset.

16. The method of claim 15, wherein the method comprises obtaining the US image data or PA image data by translating the multi-modal imaging probe along an elevation (y) direction, for which 2D or 3D US imaging, 2D or 3D PA imaging or 2D or 3D US imaging is performed sequentially by moving the multi-modal imaging probe in increments.

17. The method of claim 15, wherein the method comprises generating the multimodal image data by performing the intermodal image registration of the FL image data and one of US coronal plane (C-plane) image data, PA C-plane image data or combined US/PA C-plane image data.

18. The method of claim 17, wherein the combined US/PA C-plane image data are in multiple cross-sectional planes formed by lateral and axial axes, the FL image data are in a second C-plane at a given depth and the intermodal registration is based on using the multiple cross-sectional US/PA C-plane image data at the given depth to construct a C-plane US/PA image and onto which the FL image data is overlaid.

19. The method of claim 17, wherein the method comprises generating the combined US/PA C-plane image data by using alpha-blending

20. The method of claim 17, wherein the method comprises and applying pixel scaling when combining FL image data with US image data, PA image data or combined US/PA image data.
